(19) United States
(12) Patent Application Publication
(10) Pub. No.: US 2005/0070529 A1
(43) Pub. Date: Mar. 31, 2005

(54) Use of type 4 phosphodiesterase inhibitors in myocardial diseases

(75) Inventors: Arne Sutter, Muehltal (DE); Thomas Ehring, Remscheid (DE); Thomas Welge, Alsbach (DE); Klause Minck, Ober-Ramstadt (DE); Claudia Wilm, Darmstadt (DE); Michael Gassen, Muenchen (DE); Hans-Michael Eggenweiler, Darmstadt (DE); Michael Wolf, Darmstadt (DE); Pierre Schelling, Muehltal (DE); Norbert Beier, Reinheim (DE); Joachim Leibrock, Pfungstadt (DE)

Correspondence Address: MILLEN, WHITE, ZELANO & BRANIGAN, P.C., 2200 CLARENDON BLVD., SUITE 1400, ARLINGTON, VA, 22201, US

(73) Assignee: Merk Pantent GmbH, Darmstadt (DE)

(21) Appl. No.: 10/467,793

(22) PCT Filed: Jan. 15, 2002
(86) PCT No.: PCT/EP02/00320

(30) Foreign Application Priority Data
Feb. 12, 2001 (EP) 01102811.5
Aug. 17, 2001 (DE) 01119875.1

(51) Int. Cl. A61K 31/549; A61K 31/501; A61K 31/50

(52) U.S. Cl. 514/222.5; 514/247; 514/252.03

(57) ABSTRACT

The invention relates to the use of type 4 phosphodiesterase inhibitors to treat myocardial diseases.

The invention relates to the use of type 4 phosphodiesterase inhibitors to treat myocardial diseases.

Coronary artery disease is the most common cause of death in the western world. In the presence of a critical stenosed coronary artery, a decrease of blood flow may result in myocardial ischemia. Initiation of reperfusion results, depending on the severity of the preceding ischemic period, in a reversibly or irreversibly injured myocardium, which is characterized by a long-lasting depression or an irreversible loss of contractile function. Depending on the size of the affected myocardial area, an acute or a chronic heart failure may develop.

A particular clinical problem in the above mentioned scenario is the development of restenosis after a primarily successful reperfusion intervention by PTCA, even after stent implantation, thrombolysis or coronary artery bypass grafting.

From experimental animal and clinical studies evidence exists, that in the different above mentioned heart diseases, i. e. coronary artery disease itself, reversible or irreversible myocardial ischemia/reperfusion injury, acute or chronic heart failure and restenosis, including instent-restenosis and stent-in-stent-restenosis, inflammatory processes play a casual role. These inflammatory processes involve resident and invading macrophages as well as neutrophils and TH₁ and TH₂ helper cells. This leukocyte response produces the characteristic cytokine pattern, involving TNF-α, IL-1β, IL-2, and IL-6, as well as IL-10 and IL-13 (Pulkki K J: Cytokines and cardiomyocyte death. Ann.Med. 1997 29: 339-343.

Birks E J, Yacoub M H: The role of nitric oxide and cytokines in heart failure. Coron.Artery.Dis. 1997 8: 389-402).

The formation of these species has been demonstrated in human patients with myocardial ischemia. Animal models show that cytokine production correlates with the invasion of peripheral macrophages and neutrophils which can spread the damage into the still intact myocardium.

The main player in the cytokine response, however, is TNF-a, which integrates inflammatory and pro-apoptotic responses and additionally has a direct negative ionotropic effect on cardiac myocytes (Ceconi C, Curello S, Bachetti T, Corti A, Ferrari R: Tumor necrosis factor in congestive heart failure: a mechanism of disease for the new millennium?

Prog.Cardiovasc.Dis. 1998 41: 25-30.

Mann D L: The effect of tumor necrosis factor-alpha on cardiac structure and function: a tale of two cytokines. J.Card.Fail. 1996 2: S165-S172.

Squadrito F, Altavilla D, Zingarelli B, et al: Tumor necrosis factor involvement in myocardial ischaemia-reperfusion injury. Eur.J.Pharmacol. 1993 237: 223-230).

It has been shown in animal models of myocardial infarction, that TNF-α is rapidly released during the reperfusion phase (Herskowitz A, Choi S, Ansari A A, Wesselingh S: Cytokine mRNA expression in postischemic/reperfused myocardium. Am.J.Pathol. 1995 146: 419-428) and that the protective effects of drugs such as dexamethasone (Arras M, Strasser R, Mohri M, et al: Tumor necrosis factor-alpha is expressed by monocytes/macrophages following cardiac microembolization and is antagonized by cyclosporine. Basic.Res.Cardiol. 1998 93: 97-107), cyclosporin A (Arras M, Strasser R, Mohri M, et al: Tumor necrosis factor-alpha is expressed by monocytes/macrophages following cardiac microembolization and is antagonized by cyclosporine. Basic.Res.Cardiol. 1998 93: 97-107.

Squadrito F, Altavilla D, Squadrito G, et al: Cyclosporin-A reduces leukocyte accumulation and protects against myocardial ischaemia reperfusion injury in rats. Eur.J.Pharmacol. 1999 364: 159-168) or clorichromene (Squadrito F. Altavilla D, Zingarelli B, et al: The effect of cloricromene, a coumarine derivative, on leukocyte accumulation, myocardial necrosis and TNF-alpha production in myocardial ischaemia-reperfusion injury. Life Sci. 1993 53: 341-355)correspond with a reduction of circulating TNF-α.

Preferred PDE4 inhibitors mentioned below are potent antagonists of macrophage and T-cell cytokine production. They also inhibit the proliferation of T cells. Consequently, PDE4 inhibition may have a beneficial effect in those myocardial diseases, which are causally linked to cytokine production and inflammatory processes.

As compared with PDE3 inhibitors and the early PDE4 inhibitor Rolipram, preferred PDE4 inhibitors are devoid of hemodynamic side effects, which can be dose limiting for the treatment of most cardiovascular disorders.

The invention was based on the object of discovering new uses of compounds having valuable properties, especially those which may be used to prepare medicaments.

It has been found that the preferred PDE IV inhibitors and their salts combine very valuable pharmacological properties with good tolerability for the treatment of myocardial diseases.

Accordingly, the invention provides in particular for the use of
a) compounds of formula l disclosed in EP 0763534 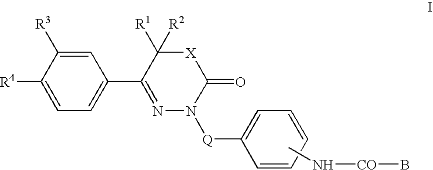
   in which
B is an aromatic heterocycle having 1 to 4 N, O and/or S atoms, bonded via N or C, which can be unsubstituted or mono-, di- or trisubstituted by HaI, A and/or OA, and can also be fused to a benzene or pyridine ring,
Q is absent or is alkylene having 1-6 C atoms,
X is CH₂, S or O,
R¹ and R² in each case independently of one another are H or A,
R³ and R⁴ in each case independently of one another are —OH, OR⁵, —S—R⁵, —SO—R⁵, —SO₂—R⁵, HaI, methylenedioxy, —NO₂, —NH₂, —NHR⁵ or —NR⁵R⁶,
R⁵ and R⁶ in each case independently of one another are A, cycloalkyl having 3-7 C atoms, methylenecycloalkyl having 4-8 C atoms or alkenyl having 2-8 C atoms,
A is alkyl having 1 to 10 C atoms, which can be substituted by 1 to 5 F and/or Cl atoms and
HaI is F, Cl, Br or I
   and their stereoisomers and physiologically acceptable, salts and solvates;
b) compounds of formula I disclosed in WO 99/65880 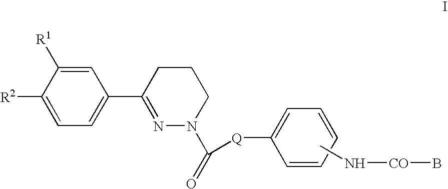
   in which
B is a phenyl ring which is unsubstituted or mono- or polysubstituted by R³,
Q is absent or is alkylene having 1-4 C atoms,
R¹,R² each independently of one another are —OR⁴, —S—R⁴, —SO—R⁴, —SO₂—R⁴ or HaI,
R¹ and R² together are also —O—CH₂—O—,
R³ is R⁴, HaI, OH, OR⁴, OPh, NO₂, NHR⁴, N(R⁴)₂, NHCOR⁴, NHSO₂R⁴ or NHCOOR⁴,
R⁴ is A, cycloalkyl having 3-7 C atoms, alkylenecycloalkyl having 5-10 C atoms or alkenyl having 2-8 C atoms,
A is alkyl having 1 to 10 C atoms, which can be substituted by 1 to 5 F and/or Cl atoms and
HaI is F, Cl, Br or I,
   and their physiologically acceptable salts and solvates;
c) compounds of formula I disclosed in WO 00/26201 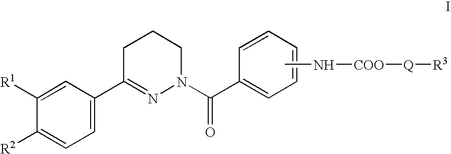
   in which
R¹, R² in each case independently of one another are —OH, OR⁵, —S—R⁵, —SO—R⁵, —SO₂—R⁵ or HaI,
R¹ and R² together are also —O—CH₂—O—,
R³ is NH₂, NHA, NAA′ or a saturated heterocycle having 1 to 4 N, O and/or S atoms which can be unsubstituted or mono-, di- or tri-substituted by HaI, A and/or OA,
Q is absent or is branched or unbranched alkylene having 1-10 C atoms,
R⁵ is A, cycloalkyl having 3-7 C atoms, alkylenecycloalkyl having 4-8 C atoms or alkenyl having 2-8 C atoms,
A, A′ in each case independently of one another are alkyl which has 1 to 10 C atoms and which can be substituted by 1 to 5 F and/or Cl atoms and
HaI is F, Cl, Br or I,
   and the physiologically acceptable salts and solvates thereof;
d) compounds of formula I disclosed in WO 98/06704 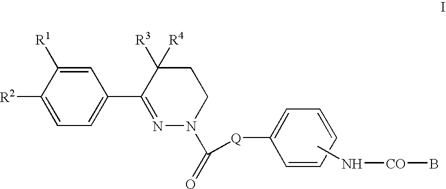
   in which
B is A, OA, NH₂, NHA, NAA′ or an unsaturated heterocycle which has 1 to 4 N, O and/or S atoms and which can be unsubstituted or mono-, di- or trisubstituted by HaI, A and/or OA,
Q is absent or is alkylene having 1-6 C atoms,
R¹, R² in each case independently of one another are —OH, OR⁵, —S—R⁵, —SO—R⁵, —SO₂—R⁵, HaI, —NO₂, —NH₂, —NHR⁵ or —NR⁵R⁶,
R¹ and R² together are also —O—CH₂—O—,
R³ R⁴ in each case independently of one another are H or A,
R⁵, R⁶ in each case independently of one another are A, cycloalkyl having 3-7 C atoms, methylenecycloalkyl having 4-8 C atoms or alkenyl having 2-8 C atoms,
A, A′ in each case independently of one another are alkyl which has 1 to 10 C atoms and which can be substituted by 1 to 5 F and/or Cl atoms and
HaI is F, Cl, Br or I,
   and the stereoisomers and physiologically acceptable salts and solvates thereof;
e) compounds disclosed in WO 00/59890
   1-(4-ureidobenzoyl)-3-(3-ethoxy-4-methoxyphenyl)-1,4,5,6-tetrahydropyridazine,
   1-(4-nicotinoylaminobenzoyl)-3-(3-propoxy-4-methoxyphenyl)-1,4,5,6-tetrahydropyridazine,
   1-(4-trifluoroacetamidobenzoyl)-3-(3-ethoxy-4-methoxyphenyl)-1,4,5,6-tetrahydropyridazine,
   1-(4-ethoxycarbonylaminobenzoyl)-3-(3-propoxy-4-methoxyphenyl)-1,4,5,6-tetrahydropyridazine,
   1-(4-isopropoxycarbonylaminobenzoyl)-3-(3-ethoxy-4-methoxyphenyl)-1,4,5,6-tetrahydropyridazine,
   1-(4-propoxycarbonylaminobenzoyl)-3-(3ethoxy-4-methoxyphenyl)-1,4,5,6-tetrahydropyridazine,
   1-(4-nicotinoylaminobenzoyl)-3-(3,4-dimethoxyphenyl)-4-ethyl-1,4,5,6-tetrahydropyridazine,
   1-(4-ethoxycarbonylaminobenzoyl)-3-(3,4-dimethoxyphenyl)-4-ethyl-1,4,5,6-tetrahydropyridazine and
   1-(4-acetamidobenzoyl)-3-(3,4-dimethoxyphenyl)-4-ethyl-1,4,5,6-tetrahydropyridazine,
      and their physiologically acceptable salts and solvates;
f) compounds of formula I disclosed in DE 19604388 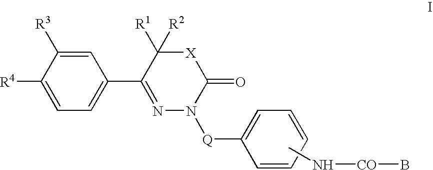
   in which
R¹, R² in each case independently of one another are H or A,
R³, R⁴ in each case independently of one another are —OH, OA, —S-A, —SO-A, —SO₂-A, HaI, methylenedioxy, —NO₂, —NH₂, —NHA or —NAA′,
A, A′ in each case independently of one another are alkyl having 1 to 10 C-atoms, and which can be substituted by 1 to 5 F and/or Cl atoms, cycloalkyl having 3-7 C atoms or methylenecycloalkyl having 4-8 C atoms,
B is —Y—R⁵ oder —O—Y—R⁵,
Q is absent or is alkylene having 1-4 C atoms,
Y is absent or is alkylene having 1-10 C atoms,
X is CH₂ or S.
R⁵ is NH₂, NHA, NAA′ or is a saturated 3-8 membered heterocycle having at least one N atom, and wherein other CH₂ groups optionally may be replaced by NH, NA, S or O, which can be unsubstituted or monosubstituted by A or OH,
HaI is F, Cl, Br oder I
   and the stereoisomers and physiologically acceptable salts and solvates thereof;
g) compounds of formula I disclosed in DE 19932315 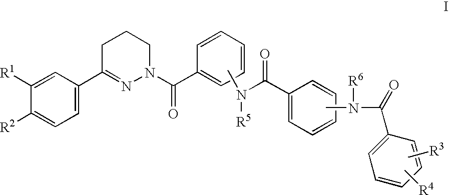
   in which
R¹, R² in each case independently of one another are H, OH, OA, SA, SOA, SO₂A, F, Cl or A′₂N—(CH₂)ₙ—O—,
R¹ and R² together are also —O—CH₂—O—,
R³, R⁴ in each case independently of one another are H, A, HaI, OH, OA, NO₂, NHA, NA₂, CN, COOH, COOA, NHCOA, NHSO₂A or NHCOOA,
R⁵, R⁶ in each case independently of one another are H or alkyl having 1 to 6 C atoms,
A is alkyl having 1 to 10 C atoms, which can be substituted by 1 to 5 F and/or Cl atoms,
   is cycloalkyl having 3-7 C atoms, alkylenecycloalkyl having 5-10 C atoms or alkenyl having 2-8 C atoms,
A′ is alkyl having 1, 2, 3, 4, 5 or 6 C atoms,
n is 1, 2, 3 or 4,
HaI is F, Cl, Br or 1,
   and their physiologically acceptable salts and solvates;
h) compounds of formula I disclosed in EP 0723962 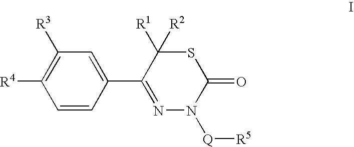
   in which
R¹ and R² in each case independently of one another are H or A,
R³ and R⁴ in each case independently of one another are —OH, —OR¹⁰, —S—R¹⁰, —SO—R¹⁰, —SO₂R¹°, HaI, methylenedioxy, —NO₂, —NH₂, —NHR¹⁰ or —NR¹⁰R¹¹,
R⁵ is a phenyl radical which is unsubstituted or mono- or disubstituted by R⁶ and/or R⁷,
Q is absent or is alkylene having 1-6 C atoms,
R⁶ and R⁷ in each case independently of one another are —NH₂, —NR⁸R⁹, —NHR¹⁰, —NR¹⁰R¹¹, —NO₂, HaI, —CN, —OA, —COOH or —COOA,
R⁸ and R⁹ in each case independently of one another are H, acyl having 1-8 C atoms which can be substituted by 1-5 F and/or Cl atoms, —COOA, —S-A, —SO-A, —SO₂A, —CONH₂, —CONHA, —CONA₂, —CO—COOH, —CO—COOA, —CO—CONH₂, —CO—CONHA or —CO—CONA₂.
A is alkyl having 1 to 6 C atoms which can be substituted by 1-5 F and/or Cl atoms,
R¹⁰ and R¹¹ in each case independently of one another are A, cycloalkyl having 3-7 C atoms, methylenecycloalkyl having 4-8 C atoms or alkenyl having 2-8 C-atoms
   and
HaI is F, Cl, Br or I,
   and their physiologically acceptable salts and solvates;
i) compounds of formula I disclosed in EP 0738715 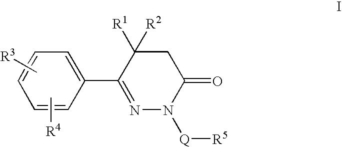
   in which
R¹ and R² in each case independently of one another are H or A,
R³ and R⁴ in each case independently of one another are —OH, —OR¹⁰, —S—R¹⁰, —SO—R¹⁰, —SO₂R¹⁰, HaI, methylenedioxy, —NO₂, —NH₂, —NHR¹⁰ or —NR¹⁰R¹¹,
R⁵ is a phenyl radical which is unsubstituted or mono- or disubstituted by R⁶ and/or R⁷,
Q is absent or is alkylene having 1-6 C atoms,
R⁶ and R⁷ in each case independently of one another are —NH₂, —NR⁸R⁹, —NHR¹⁰, —NR¹⁰R¹¹, —NO₂, HaI, —CN, —OA, —COOH or —COOA,
R⁸ and R⁹ in each case independently of one another are H, acyl having 1-8 C atoms which can be substituted by 1-5 F and/or Cl atoms, —COOA, —SO-A, —SO₂A, —CONH₂, —CONHA, —CONA₂, —CO—COOH, —CO—COOA, —CO—CONH₂, —CO—CONHA or —CO—CONA₂,
A is alkyl having 1 to 6 C atoms which can be substituted by 1-5 F and/or Cl atoms,
R¹⁰ and R¹¹ in each case independently of one another are A, cycloalkyl having 3-7 C atoms, methylenecycloalkyl having 4-8 C atoms or alkenyl having 2-8 C-atoms
   and
HaI is F, Cl, Br or I,
   and their physiologically acceptable salts and solvates;
   for preparing a medicament for treating myocardial diseases.

Preferably, the invention provides for the use of
a) compounds disclosed in EP 0763534:
   2-(3-nicotinoylaminobenzyl)-6-(3,4-dimethoxyphenyl)-2,3,4,5-tetrahydropyridazin-3-one,
   2-(2-nicotinoylaminobenzyl)-6-(3,4-dimethoxyphenyl)-2,3,4,5-tetrahydropyridazin-3-one,
   2-(4-nicotinoylaminobenzyl)-6-(3,4-dimethoxyphenyl)-5-ethyl-2,3,4,5-tetrahydropyridazin-3-one,
   2-(3-nicotinoylaminobenzyl)-6-(3,4-dimethoxyphenyl)-5-ethyl-2,3,4,5-tetrahydropyridazin-3-one,
   2-(2-nicotinoylaminobenzyl)-6-(3,4-dimethoxyphenyl)-5-ethyl-2,3,4,5-tetrahydropyridazin-3-one,
   2-(4-nicotinoylaminobenzyl)-6-(3-methoxy-4-trifluoromethoxyphenyl)-5-ethyl-2,3,4,5-tetrahydropyridazin-3-one,
   2-(4-nicotinoylaminobenzyl)-6-(3-methoxy-4-difluoromethoxyphenyl)-5-ethyl-2,3,4,5-tetrahydropyridazin-3-one,
   2-(4-nicotinoylaminobenzyl)-6-(3-methoxy-4-fluoromethoxyphenyl)-5-ethyl-2,3,4,5-tetrahydropyridazin-3-one,
   2-(4-nicotinoylaminobenzyl)-6-(3-difluoromethoxy-4-methoxyphenyl)-5-ethyl-2,3,4,5-tetrahydropyridazin-3-one,
   2-(4-nicotinoylaminobenzyl)-6-(3-trifluoromethoxy-4-methoxyphenyl)-5-ethyl-2,3,4,5-tetrahydropyridazin-3-one,
   2-(4-nicotinoylaminobenzyl)-6-(3-fluoromethoxy-4-methoxyphenyl)-5-ethyl-2,3,4,5-tetrahydropyridazin-3-one,
   2-(4-nicotinoylaminobenzyl)-6-(3-methoxy-4-ethoxyphenyl)-5-ethyl-2,3,4,5-tetrahydropyridazin-3-one,
   2-(4-nicotinoylaminobenzyl)-4-(3-ethoxy-4-methoxyphenyl)-5-ethyl-2,3,4,5-tetrahydropyridazin-3-one,
   2-(4-nicotinoylaminobenzyl)-6-(3-hydroxy-4-methoxyphenyl)-5-ethyl-2,3,4,5-tetrahydropyridazin-3-one,
2-(4-nicotinoylaminobenzyl)-6-(4-methylsulfonylphenyl)-5ethyl-2,3,4,5-tetrahydropyridazin-3-one,
2-(4-nicotinoylaminobenzyl)-6-(4-methyleneoxyphenyl)-5-ethyl-2,3,4,5-tetrahydropyridazin-3-one,
   2-(4-nicotinoylaminobenzyl)-6-(3-cyclopentyloxy-4-methoxyphenyl)-5-ethyl-2,3,4,5-tetrahydropyridazin-3-one,
   2-(3-nicotinoylaminobenzyl)-6-(3-cyclopentyloxy-4-methoxyphenyl)-5-ethyl-2,3,4,5-tetrahydropyridazin-3-one,
   2-(4-nicotinoylaminophenethyl)-6-(3,4-dimethoxyphenyl)-2,3,4,5-tetrahydropyridazin-3-one,
   2-(4-nicotinoylaminophenethyl)-6-(3,4-dimethoxyphenyl)-5-ethyl-2,3,4,5-tetrahydropyridazin-3-one,
   3-(4-nicotinoylaminobenzyl)-5-(3,4-dimethoxyphenyl)-3,6-dihydro-1,3,4-thiadiazin-2-one,
   3-(3-nicotinoylaminobenzyl)-5-(3,4-dimethoxyphenyl)-3,6-dihydro-1,3,4-thiadiazin-2-one,
   3-(2-nicotinoylaminobenzyl)-5-(3,4-dimethoxyphenyl)-3,6-dihydro-1,3,4-thiadiazin-2-one,
   3-(4-nicotinoylaminobenzyl)-5-(3,4-dimethoxyphenyl)-6-ethyl-3,6-dihydro-1,3,4-thiadiazin-2-one,
   3-(3-nicotinoylaminobenzyl)-5-(3,4-dimethoxyphenyl)-6-ethyl-3,6-dihydro-1,3,4-thiadiazin-2-one,
   3-(2-nicotinoylaminobenzyl)-5-(3,4-dimethoxyphenyl)-6-ethyl-3,6-dihydro-1,3,4-thiadiazin-2-one,
   3-(4-nicotinoylaminobenzyl)-5-(3-methoxy-4-trifluoromethoxyphenyl)-6-ethyl-3,6-dihydro-1,3,4-thiadiazin-2-one,
   3-(4-nicotinoylaminobenzyl)-5-(3-methoxy-4-difluoromethoxyphenyl)-6-ethyl-3,6-dihydro-1,3,4-thiadiazin-2-one,
   3-(4-nicotinoylaminobenzyi)-5-(3-methoxy-4-fluoromethoxyphenyl)-6-ethyl-3,6-dihydro-1,3,4-thiadiazin-2-one,
   3-(4-nicotinoylaminobenzyl)-5-(3-difluoromethoxy-4-methoxyphenyl)-6-ethyl-3,6-dihydro-1,3,4-thiadiazin-2-one,
   3-(4-nicotinoylaminobenzyl)-5-(3-trifluoromethoxy-4-methoxyphenyl)-6-ethyl-3,6-dihydro-1,3,4-thiadiazin-2-one,
   3-(4-nicotinoylaminobenzyl)-5-(3-fluoromethoxy-4-methoxyphenyl)-6-ethyl-3,6-dihydro-1,3,4-thiadiazin-2-one,
   3-(4-nicotinoylaminobenzyl)-5-(3-methoxy-4-ethoxyphenyl)-6-ethyl-3,6-dihydro-1,3,4-thiadiazin-2-one,
   3-(4-nicotinoylaminobenzyl)-5-(3-ethoxy-4-methoxyphenyl)-6-ethyl-3,6-dihydro-1,3,4-thiadiazin-2-one,
   3-(4-nicotinoylaminobenzyl)-5-(3-ethoxy-4-methoxyphenyl)-3,6-dihydro-1,3,4-thiadiazin-2-one,
   3-(4-nicotinoylaminobenzyl)-5-(3-hydroxy-4-methoxyphenyl)-6-ethyl-3,6-dihydro-1,3,4-thiadiazin-2-one,
   3-(4-nicotinoylaminobenzyl)-5-(4-methysulfonylphenyl)-6-ethyl-3,6-dihydro-1,3,4-thiadiazin-2-one,
   3-(4-nicotinoylaminobenzyl)-5-(4-methyleneoxyphenyl)-6-ethyl-3,6-dihydro-1,3,4-thiadiazin-2-one,
   3-(4-nicotinoylaminobenzyl)-5-(3-cyclopentyloxy-4-methoxyphenyl)-6-ethyl-3,6-dihydro-1,3,4-thiadiazin-2-one,
   3-(3-nicotinoylaminobenzyl)-5-(3-cyclopentyloxy-4-methoxyphenyl)-6-ethyl-3,6-dihydro-1,3,4-thiadiazin-2-one,
   3-(4-nicotinoylaminophenethyl)-5-(3,4-dimethoxyphenyl)-3,6-dihydro-1,3,4-thiadiazin-2-one,
   3-(4-nicotinoylaminophenethyl)-5-(3,4-dimethoxyphenyl)-6-ethyl-3,6-dihydro-1,3,4-thiadiazin-2-one,
   3-(4-nicotinoylaminobenzyl)-5-(3,4-dimethoxyphenyl)-3,6-dihydro-1,3,4-oxadiazin-2-one,
   3-(3-nicotinoylaminobenzyl)-5-(3,4-dimethoxyphenyl)-3,6-dihydro-1,3,4-oxadiazin-2-one,
   3-(2-nicotinoylaminobenzyl)-5-(3,4-dimethoxyphenyl)-3,6-dihydro-1,3,4-oxadiazin-2-one,
   3-(4-nicotinoylaminobenzyl)-5-(3,4-dimethoxyphenyl)-6-ethyl-3,6-dihydro-1,3,4-oxadiazin-2-one,
   3-(3-nicotinoylaminobenzyl)-5-(3,4-dimethoxyphenyl)-6-ethyl-3,6-dihydro-1,3,4-oxadiazin-2-one,
   3-(2-nicotinoylaminobenzyl)-5-(3,4-dimethoxyphenyl)-6-ethyl-3,6-dihydro-1,3,4-oxadiazin-2-one,
   3-(4-nicotinoylaminobenzyl)-5-(3-methoxy-4-trifluoromethoxyphenyl)-6-ethyl-3,6-dihydro-1,3,4-oxadiazin-2-one,
   3-(4-nicotinoylaminobenzyl)-5-(3-methoxy-4-difluoromethoxyphenyl)-6-ethyl-3,6-dihydro-1,3,4-oxadiazin-2-one,
   3-(4-nicotinoylaminobenzyl)-5-(3-methoxy-4-fluoromethoxyphenyl)-6-ethyl-3,6-dihydro-1,3,4-oxadiazin-2-one,
   3-(4-nicotinoylaminobenzyl)-5-(3-difluoromethoxy-4-methoxyphenyl)-6-ethyl-3,6-dihydro-1,3,4-oxadiazin-2-one,
   3-(4-nicotinoylaminobenzyl)-5-(3-trifluoromethoxy-4-methoxyphenyl)-6-ethyl-3,6-dihydro-1,3,4-oxadiazin-2-one,
   3-(4-nicotinoylaminobenzyl)-5-(3-fluoromethoxy-4-methoxyphenyl)-6-ethyl-3,6-dihydro-1,3,4-oxadiazin-2-one,
   3-(4-nicotinoylaminobenzyl)-5-(3-methoxy-4-ethoxyphenyl)-6-ethyl-3,6-dihydro-1,3,4-oxadiazin-2-one,
   3-(4-nicotinoylaminobenzyl)-5-(3-ethoxy-4-methoxyphenyl)-6-ethyl-3,6-dihydro-1,3,4-oxadiazin-2-one,
   3-(4-nicotinoylaminobenzyl)-5-(3-hydroxy-4-methoxyphenyl)-6-ethyl-3,6-dihydro-1,3,4-oxadiazin-2-one,
   3-(4-nicotinoylaminobenzyl)-5-(4-methylsulfonylphenyl)-6-ethyl-3,6-dihydro-1,3,4-oxadiazin-2-one,
   3-(4-nicotinoylaminobenzyl)-5-(4-methyleneoxyphenyl)-6-ethyl-3,6-dihydro-1,3,4-oxadiazin-2-one,
   3-(4-nicotinoylaminobenzyl)-5-(3-cyclopentyloxy-4-methoxyphenyl)-6-ethyl-3,6-dihydro-1,3,4-oxadiazin-2-one,
   3-(3-nicotinoylaminobenzyl)-5-(3-cyclopentyloxy-4-methoxyphenyl)-6-ethyl-3,6-dihydro-1,3,4-oxadiazin-2-one,
   3-(4-nicotinoylaminophenethyl)-5-(3,4-dimethoxyphenyl)-3,6-dihydro-1,3,4-oxadiazin-2-one,
   3-(4-nicotinoylaminophenethyl)-5-(3,4-dimethoxyphenyl)-6-ethyl-3,6-dihydro-1,3,4-oxadiazin-2-one,
   2-(3-nicotinoylaminobenzyl)-6-(3-ethoxy-4-methoxyphenyl)-2,3,4,5-tetrahydropyridazin-3-one,
   2-(4-isonicotinoylaminobenzyl)-6-(3-ethoxy-4-methoxyphenyl)-2,3,4,5-tetrahydropyridazin-3-one,
   2-(4-pyrazinecarbonylaminobenzyl)-6-(3-ethoxy-4-methoxyphenyl)-2,3,4,5-tetrahydropyridazin-3-one,
   2-(4-(isoxazole-5-carbonylamino)benzyl)-6-(3-ethoxy-4-methoxyphenyl)-2,3,4,5-tetrahydropyridazin-3-one,
   2-(4-nicotinoylaminobenzyl)-6-(3-cyclopentyloxy-4-methoxyphenyl)-2,3,4,5-tetrahydropyridazin-3-one,
   2-(4-nicotinoylaminobenzyl)-6-(3,4,-dimethoxyphenyl)-2,3,4,5-tetrahydropyridazin-3-one, hydrochloride,
      and their stereoisomers and physiologically acceptable, salts and solvates;
b) compounds disclosed in WO 99/65880
   N-(3-(3-ethoxy-4-methoxyphenyl)-1,4,5,6-tetrahydropyridazin-1-ylcarbonyl)phenyl)-4-methoxybenzoyl-3-carboxamide,
   N-(3-(3-ethoxy-4-methoxyphenyl)-1,4,5,6-tetrahydropyridazin-1-ylcarbonyl)phenyl)-4-methylbenzoyl-3-carboxamide,
   N-(3-(3-ethoxy-4-methoxyphenyl)-1,4,5,6-tetrahydropyridazin-1-ylcarbonyl)phenyl)benzoyl-3-carboxamide,
   N-(3-(3-ethoxy-4-methoxyphenyl)-1,4,5,6-tetrahydropyridazin-1-ylcarbonyl)phenyl)-3,4-dichlorobenzoyl-3-carboxamide,
   N-(3-(3-ethoxy-4-methoxyphenyl)-1,4,5,6-tetrahydropyridazin-1-ylcarbonyl)phenyl)-4-trifluoromethylbenzoyl-3-carboxamide,
   N-(3-(3-ethoxy-4-methoxyphenyl)-1,4,5,6-tetrahydropyridazin-1-ylcarbonyl)phenyl)-3-chlorobenzoyl-3-carboxamide,
   N-(3-(3-ethoxy-4-methoxyphenyl)-1,4,5,6-tetrahydropyridazin-1-ylcarbonyl)phenyl)-4-fluorobenzoyl-3-carboxamide,
   N-(3-(3-ethoxy-4-methoxyphenyl)-1,4,5,6-tetrahydropyridazin-1-ylcarbonyl)phenyl)-4-butoxybenzoyl-3-carboxamide,
   N-(3-(3-ethoxy-4-methoxyphenyl)-1,4,5,6-tetrahydropyridazin-1-ylcarbonyl)phenyl)-4-pentoxybenzoyl-3-carboxamide,
   N-(3-(3-ethoxy-4-methoxyphenyl)-1,4,5,6-tetrahydropyridazin-1-ylcarbonyl)phenyl)-4-ethoxybenzoyl-3-carboxamide,
   N-(3-(3-ethoxy-4-methoxyphenyl)-1,4,5,6-tetrahydropyridazin-1-ylcarbonyl)phenyl)-3,4-dimethoxybenzoyl-3-carboxamide,
   N-(3-(3-ethoxy-4-methoxyphenyl)-1,4,5,6-tetrahydropyridazin-1-ylcarbonyl)phenyl)-3-methylbenzoyl-3-carboxamide,
   N-(3-(3-ethoxy-4-methoxyphenyl)-1,4,5,6-tetrahydropyridazin-1-ylcarbonyl)phenyl)-3-methoxybenzoyl-3-carboxamide,
      and their physiologically acceptable salts and solvates;
c) compounds disclosed in WO 00/26201
   3-dimethylaminopropyl {4-[3-(3-ethoxy-4-methoxyphenyl)-1,2,3,4-tetrahydropyridazin-1-ylcarbonyl]phenyl}carbamate,
   N-methylpiperidin-4-yl-4-[3-(3-ethoxy-4-methoxyphenyl)-1,2,3,4-tetrahydropyridazin-1-ylcarbonyl]phenyl}carbamate,
   3-dimethylaminopropyl {4-[3-(3-isopropoxy-4-methoxyphenyl)-1,2,3,4-tetrahydropyridazin-1-ylcarbonyl]phenyl}carbamate,
   3-dimethylaminopropyl {3-[3-(3-ethoxy-4-methoxyphenyl)-1,2,3,4-tetrahydropyridazin-1-ylcarbonyl]phenyl}carbamate,
   3-dimethylaminopropyl{3-[3-(3-cyclopentyloxy-4-methoxyphenyl)-1,2,3,4-tetrahydropyridazin-1-ylcarbonyl]phenyl}carbamate,
   N-methylpiperidin-4-yl-3[3-(3-cyclopentyloxy-4-methoxyphenyl)-1,2,3,4-tetrahydropyridazin-1-ylcarbonyl]phenyl}carbamate,
   3-dimethylaminopropyl{3-[3-(3-propyloxy-4-methoxyphenyl)-1,2,3,4-tetrahydropyridazin-1-ylcarbonyl]phenyl}carbamate,
   3-dimethylaminopropyl{4-[3-(3,4-diethoxyphenyl)-1,2,3,4-tetrahydropyridazin-1-ylcarbonyl]phenyl}carbamate,
   N-methylpiperidin-4-yl-{4-[3-(3,4-diethoxyphenyl)-1,2,3,4-tetrahydropyridazin-1-ylcarbonyl]phenyl}carbamate,
   3-dimethylaminopropyl{3-[3-(3,4-dimethoxyphenyl)-1,2,3,4-tetrahydropyridazin-1-ylcarbonyl]phenyl}carbamate
   3-dimethylaminopropyl{4-[3-(3,4-dimethoxyphenyl)-1,2,3,4-tetrahydropyridazin-1-ylcarbonyl]phenyl}carbamate,
      and the physiologically acceptable salts and solvates thereof;
d) compounds disclosed in WO 98/06704
   1-(4-nicotinoylaminobenzoyl)-3-(3,4-dimethoxyphenyl)-1,4,5,6-tetrahydropyridazine,
   1-(3-nicotinoylaminobenzoyl)-3-(3,4-dimethoxy-phenyl)-1,4,5,6-tetrahydropyridazine hydrochloride,
   1-(2-nicotinoylaminobenzoyl)-3-(3,4-dimethoxy-phenyl)-1,4,5,6-tetrahydropyridazine,
   1-(4-nicotinoylaminobenzoyl)-3-(3-ethoxy-4-methoxyphenyl)-1,4,5,6-tetrahydropyridazine,
   1-(3-nicotinoylaminobenzoyl)-3-(3-ethoxy-4-methoxyphenyl)-1,4,5,6-tetrahydropyridazine,
   1-(4-nicotinoylaminobenzoyl)-3-(3-cyclopentyloxy-4-methoxyphenyl)-1,4,5,6-tetrahydropyridazine,
   1-(3-nicotinoylaminobenzoyl)-3-(3-cyclopentyloxy-4-methoxyphenyl)-1,4,5,6-tetrahydropyridazine,
   1-(4-nicotinoylaminobenzoyl)-3-(3,4-methylene-dioxyphenyl)-1,4,5,6-tetrahydropyridazine,
   1-(4-nicotinoylaminobenzoyl)-3-(3-methoxy-4-methylsulfonylphenyl)-1,4,5,6-tetrahydro-pyridazine,
   1-(4-nicotinoylaminobenzoyl)-3-(3-trifluoro-methoxy-4-methoxyphenyl)-1,4,5,6-tetrahydro-pyridazine,
   1-(4-ethoxy-carbonylaminobenzoyl)-3-(3,4-dimethoxyphenyl)-1,4,5,6-tetrahydropyridazine,
   1-(3-ethoxycarbonylaminobenzoyl)-3-(3,4-dimethoxy-phenyl)-1,4,5,6-tetrahydropyridazine,
   1-(2-ethoxycarbonylaminobenzoyl)-3-(3,4-dimethoxy-phenyl)-1,4,5,6-tetrahydropyridazine,
   1-(4-ethoxycarbonylaminobenzoyl)-3-(3-ethoxy-4-methoxyphenyl)-1,4,5,6-tetrahydropyridazine,
   1-(3-ethoxycarbonylaminobenzoyl)-3-(3-ethoxy-4-methoxyphenyl)-1,4,5,6-tetrahydropyridazine,
   1-(4-ethoxycarbonylaminobenzoyl)-3-(3-cyclo-pentyloxy-4-methoxyphenyl)-1,4,5,6-tetrahydro-pyridazine,
   1-(3-ethoxycarbonylaminobenzoyl)-3-(3-cyclo-pentyloxy-4-methoxyphenyl)-1,4,5,6-tetrahydro-pyridazine,
   1-(4-ethoxycarbonylaminobenzoyl)-3-(3,4-methylene-dioxyphenyl)-1,4,5,6-tetrahydropyridazine,
   1-(4-ethoxycarbonylaminobenzoyl)-3-(3-methoxy-4-methylsulfonylphenyl)-1,4,5,6-tetrahydro-pyridazine,
   1-(4-ethoxycarbonylaminobenzoyl)-3-(3-trifluoro-methoxy-4-methoxyphenyl)-1,4,5,6-tetrahydro-pyridazine,
      and the stereoisomers and physiologically acceptable salts and solvates thereof;
e) compounds disclosed in EP 0723962
   3-(4-ethoxycarbonylaminobenzyl)-5-(3-ethoxy-4-methoxyphenyl)-3,6-dihydro-1,3,4-thiadiazin-2-one,
   3-(4-ethoxycarbonylaminobenzyl)-5-(3-cyclopentyloxy-4-methoxyphenyl)-3,6-dihydro-1,3,4-thiadiazin-2-one,
      and their physiologically acceptable salts and solvates;
f) compounds disclosed in EP 0738715
   2-(4-butyrylaminobenzyl)-6-(3,4-dimethoxyphenyl)-2,3,4,5-tetrahydropyridazin-3-one,
   2-(4-acetamidobenzyl)-6-(3,4-dimethoxyphenyl)-2,3,4,5-tetrahydropyridazin-3-one,
   2-(4-trifluoroacetamidobenzyl)-6-(3,4-dimethoxyphenyl)-2,3,4,5-tetrahydropyridazin-3-one,
   2-(4-methylsulfonamidobenzyl)-6-(3,4-dimethoxyphenyl)-2,3,4,5-tetrahydropyridazin-3-one,
   2-(4-propionylaminobenzyl)-6-(3,4-dimethoxyphenyl)-2,3,4,5-tetrahydropyridazin-3-one,
   2-(4-tert-butylcarbonylaminobenzyl)-6-(3,4-dimethoxyphenyl)-2,3,4,5-tetrahydropyridazin-3-one,
   2-(4-isobutyrylaminobenzyl)-6-(3,4-dimethoxyphenyl)-2,3,4,5-tetrahydropyridazin-3-one,
   2-(4-methoxycarbonylaminobenzyl)-6-(3,4-dimethoxyphenyl)-2,3,4,5-tetrahydropyridazin-3-one,
   2-(4-pivalylaminobenzyl)-6-(3,4-dimethoxyphenyl)-2, 3,4,5-tetrahydropyridazin-3-one,
   2-(4-cyclopentylcarbamoylbenzyl)-6-(3,4-dimethoxyphenyl)-2,3,4,5-tetrahydropyridazin-3-one,
   2-(4-ethoxycarbonylaminobenzyl)-6-(3,4-dimethoxyphenyl)-2, 3,4,5-tetrahydropyridazin-3-one,
   2-(4-methoxalylaminobenzyl)-6-(3,4-dimethoxyphenyl)-2,3,4,5-tetrahydropyridazin-3-one,
   2-(4-ureidobenzyl)-6-(3,4-dimethoxyphenyl)-2,3,4,5-tetrahydropyridazin-3-one,
   2-(4-pentanoylaminobenzyl)-6-(3,4-dimethoxyphenyl)-2,3,4,5-tetrahydropyridazin-3-one,
   2-(4-hexanoylaminobenzyl)-6-(3,4-dimethoxyphenyl)-2,3,4,5-tetrahydropiridazin-3-one,
   2-(4-pentafluoropropionylaminobenzyl)-6-(3,4-dimethoxyphenyl)-2,3,4,5-tetrahydropyridazin-3-one,
   2-(4-acetamidobenzyl)-6-(3,4-dimethoxyphenyl)-5-ethyl-2,3,4,5-tetrahydropyridazin-3-one,
   2-(4-trifluoroacetamidobenzyl)-6-(3,4-dimethoxyphenyl)-5-ethyl-2,3,4,5-tetrahydropyridazin-3-one,
   2-(4-methylsulfonamidobenzyl)-6-(3,4-dimethoxyphenyl)-5-ethyl-2,3,4,5-tetrahydropyridazin-3-one,
   2-(4-propionylaminobenzyl)-6-(3,4-dimethoxyphenyl)-5-ethyl-2,3,4,5-tetrahydropyridazin-3-one,
   2-(4-tert-butylcarbonylaminobenzyl)-6-(3,4-dimethoxyphenyl)-5-ethyl-2,3,4,5-tetrahydropyridazin-3-one,
   2-(4-butyrylaminobenzyl)-6-(3,4-dimethoxyphenyl)-5-ethyl-2,3,4,5-tetrahydropyridazin-3-one,
   2-(4-isobutyrylaminobenzyl)-6-(3,4-dimethoxyphenyl)-5-ethyl-2,3,4,5-tetrahydropyridazin-3-one,
   2-(4-methoxycarbonylaminobenzyl)-6-(3,4-dimethoxyphenyl)-5-ethyl-2,3,4,5-tetrahydropyridazin-3-one,
   2-(4-pivalylaminobenzyl)-6-(3,4-dimethoxyphenyl)-5-ethyl-2,3,4,5-tetrahydropyridazin-3-one,
   2-(4-cyclopentylcarbamoylbenzyl)-6-(3,4-dimethoxyphenyl)-5-ethyl-2,3,4,5-tetrahydropyridazin-3-one,
   2-(4-ethoxycarbonylaminobenzyl)-6-(3,4-dimethoxyphenyl)-5-ethyl-2,3,4,5-tetrahydropyridazin-3-one,
   2-(4-methoxalylaminobenzyl)-6-(3,4-dimethoxyphenyl)-5-ethyl-2,3,4,5-tetrahydropyridazin-3-one,
   2-(4-ureidobenzyl)-6-(3,4-dimethoxyphenyl)-5-ethyl-2,3,4,5-tetrahydropyridazin-3-one,
   2-(4-pentanoylaminobenzyl)-6-(3,4-dimethoxyphenyl)-5-ethyl-2,3,4,5-tetrahydropyridazin-3-one,
   2-(4-hexanoylaminobenzyl)-6-(3,4-dimethoxyphenyl)-5-ethyl-2,3,4,5-tetrahydropyridazin-3-one,
   2-(4-pentafluoropropionylaminobenzyl)-6-(3,4-dimethoxyphenyl)-5-ethyl-2,3,4,5-tetrahydropyridazin-3-one,
   2-(4-acetamidobenzyl)-6-(3-ethoxy-4-methoxyphenyl)-5-ethyl-2,3,4,5-tetrahydropyridazin-3-one,
   2-(4-trifluoroacetamidobenzyl)-6-(3-ethoxy-4-methoxyphenyl)-5-ethyl-2,3,4,5-tetrahydropyridazin-3-one,
   2-(4-methylsulfonamidobenzyl)-6-(3-ethoxy-4-methoxyphenyl)-5-ethyl-2,3,4,5-tetrahydropyridazin-3-one,
   2-(4-propionylaminobenzyl)-6-(3-ethoxy-4-methoxyphenyl)-5-ethyl-2,3,4,5-tetrahydropyridazin-3-one,
   2-(4-butyrylaminobenzyl)-6-(3-ethoxy-4-methoxyphenyl)-5-ethyl-2,3,4,5-tetrahydropyridazin-3-one,
   2-(4-isobutyrylaminobenzyl)-6-(3-ethoxy-4-methoxyphenyl)-5-ethyl-2,3,4,5-tetrahydropyridazin-3-one,
   2-(4-methoxycarbonylaminobenzyl)-6-(3-ethoxy-4-methoxyphenyl)-5-ethyl-2,3,4,5-tetrahydropyridazin-3-one,
   2-(4-pivalylaminobenzyl)-6-(3-ethoxy-4-methoxyphenyl)-5-ethyl-2,3,4,5-tetrahydropyridazin-3-one,
   2-(4-cyclopentylcarbamoylbenzyl)-6-(3-ethoxy-4-methoxyphenyl)-5-ethyl-2,3,4,5-tetrahydropyridazin-3-one,
   2-(4-ethoxycarbonylaminobenzyl)-6-(3-ethoxy-4-methoxyphenyl)-5-ethyl-2,3,4,5-tetrahydropyridazin-3-one;
   2-(4-methoxalylaminobenzyl)-6-(3-ethoxy-4-methoxyphenyl)-5-ethyl-2,3,4,5-tetrahydropyridazin-3-one,
   2-(4-ureidobenzyl)-6-(3-ethoxy-4-methoxyphenyl)-5-ethyl-2,3,4,5-tetrahydropyridazin-3-one,
   2-(4-pentanoylaminobenzyl)-6-(3-ethoxy-4-methoxyphenyl)-5-ethyl-2,3,4,5-tetrahydropyridazin-3-one,
   2-(4-hexanoylaminobenzyl)-6-(3-ethoxy-4-methoxyphenyl)-5-ethyl-2,3,4,5-tetrahydropyridazin-3-one,
   2-(4-pentafluoropropionylaminobenzyl)-6-(3-ethoxy-4-methoxyphenyl)-5-ethyl-2,3,4,5-tetrahydropyridazin-3-one,
   2-(4-acetamidobenzyl)-6-(3-cyclopentyloxy-4-methoxyphenyl)-5-ethyl-2,3,4,5-tetrahydropyridazin-3-one,
   2-(4-trifluoroacetamidobenzyl)-6-(3-cyclopentyloxy-4-methoxyphenyl)-5-ethyl-2,3,4,5-tetrahydropyridazin-3-one,
   2-(4-methylsulfonamidobenzyl)-6-(3-cyclopentyloxy-4-methoxyphenyl)-5-ethyl-2,3,4,5-tetrahydropyridazin-3-one,
   2-(4-propionylaminobenzyl)-6-(3-cyclopentyloxy-4-methoxyphenyl)-5-ethyl-2,3,4,5-tetrahydropyridazin-3-one,
   2-(4-tert-butyloarbonylaminobenzyl)-6-(3-cyclopentyloxy-4-methoxyphenyl)-5-ethyl-2,3,4,5-tetrahydropyridazin-3-one,
   2-(4-butyrylaminobenzyl)-6-(3-cyclopentyloxy-4-methoxyphenyl)-5-ethyl-2,3,4,5-tetrahydropyridazin-3-one,
   2-(4-isobutyrylaminobenzyl)-6(3-cyclopentyloxy-4-methoxyphenyl)-5-ethyl-2,3,4,5-tetrahydropyridazin-3-one,
   2-(4-methoxycarbonylaminobenzyl)-6-(3-cyclopentyloxy-4-methoxyphenyl)-5-ethyl-2,3,4,5-tetrahydropyridazin-3-one,
   2-(4-pivalylaminobenzyl)-6-(3-cyclopentyloxy-4-methoxyphenyl)-5-ethyl-2,3,4,5-tetrahydropyridazin-3-one,
   2-(4-cyclopentylcarbamoylbenzyl)-6-(3-cyclopentyloxy-4-methoxyphenyl)-5-ethyl-2,3,4,5-tetrahydropyridazin-3-one,
   2-(4-ethoxycarbonylaminobenzyl)-6-(3-cyclopentyloxy-4-methoxyphenyl)-5-ethyl-2,3,4,5-tetrahydropyridazin-3-one,
   2-(4-methoxalylaminobenzyl)-6-(3-cyclopentyloxy-4-methoxyphenyl)-5-ethyl-2,3,4,5-tetrahydropyridazin-3-one,
   2-(4-ureidobenzyl)-6-(3-cyclopentyloxy-4-methoxyphenyl)-5-ethyl-2,3,4,5-tetrahydropyridazin-3-one,
   2-(4-pentanoylaminobenzyl)-6-(3-cyclopentyloxy-4-methoxyphenyl)-5-ethyl-2,3,4,5-tetrahydropyridazin-3-one,
   2-(4-hexanoylaminobenzyl)-6-(3-cyclopentyloxy-4-methoxyphenyl)-5-ethyl-2,3,4,5-tetrahydropyridazin-3-one,
   2-(4-pentafluoropropionylaminobenzyl)-6-(3-cyclopentyloxy-4-methoxyphenyl)-5-ethyl-2,3,4,5-tetrahydropyridazin-3-one,
   2-(4-acetamidobenzyl)-6-(3-ethoxy-4-methoxyphenyl)-2,3,4,5-tetrahydropyridazin-3-one,
   2-(4-trifluoroacetamidobenzyl)-6-(3-ethoxy-4-methoxyphenyl)-2,3,4,5-tetrahydropyridazin-3-one,
   2-(4-methylsulfonamidobenzyl)-6-(3-ethoxy-4-methoxyphenyl)-2,3,4,5-tetrahydropyridazin-3-one,
   2-(4-propionylaminobenzyl)-6-(3-ethoxy-4-methoxyphenyl)-2,3,4,5-tetrahydropyridazin-3-one,
   2-(4-butyrylaminobenzyl)-6-(3-ethoxy-4-methoxyphenyl)-2,3,4,5-tetrahydropyridazin-3-one,
   2-(4-isobutyrylaminobenzyl)-6-(3ethoxy-4-methoxyphenyl)-2,3,4,5-tetrahydropyridazin-3-one,
   2-(4-methoxycarbonylaminobenzyl)-6-(3-ethoxy-4-methoxyphenyl)-2,3,4,5-tetrahydropyridazin-3-one,
   2-(4-pivalylaminobenzyl)-6-(3-ethoxy-4-methoxyphenyl)-2,3,4,5-tetrahydropyridazin-3-one,
   2-(4-cyclopentylcarbamoylbenzyl)-6-(3-ethoxy-4-methoxyphenyl)-2,3,4,5-tetrahydropyridazin-3-one,
   2-(4-ethoxycarbonylaminobenzyl)-6-(3-ethoxy-4-methoxyphenyl)-2,3,4,5-tetrahydropyridazin-3-one,
   2-(4-methoxalylaminobenzyl)-6-(3-ethoxy-4-methoxyphenyl)-2,3,4,5-tetrahydropyridazin-3-one,
   2-(4-ureidobenzyl)-6-(3-ethoxy-4-methoxyphenyl)-2,3,4,5-tetrahydropyridazin-3-one,
   2-(4-pentanoylaminobenzyl)-6-(3-ethoxy-4-methoxyphenyl)-2,3,4,5-tetrahydropyridazin-3-one,
   2-(4-hexanoylaminobenzyl)-6-(3-ethoxy-4-methoxyphenyl)-2,3,4,5-tetrahydropyridazin-3-one,
   2-(4-pentafluoropropionylaminobenzyl)-6-(3-ethoxy-4-methoxyphenyl)-2,3,4,5-tetrahydropyridazin-3-one,
      and their physiologically acceptable salts and solvates;
      for preparing a medicament for treating myocardial diseases.

Most preferably, the invention provides for the use of the following compounds
3-(4-nicotinoylaminobenzyl)-5-(3-ethoxy-4-methoxyphenyl)-3,6-dihydro-1,3,4-thiadiazin-2-one,
N-(3-(3-ethoxy-4-methoxyphenyl)-1,4,5,6-tetrahydropyridazin-1-ylcarbonyl)phenyl)-4-methoxybenzoyl-3-carboxamide,
1-(4-nicotinoylaminobenzoyl)-3-(3-ethoxy-4-methoxyphenyl)-1,4,5,6-tetrahydropyridazine,
1-(4-ethoxycarbonylaminobenzoyl)-3-(3-ethoxy-4-methoxyphenyl)-1,4,5,6-tetrahydropyridazine,
2-(4-ethoxycarbonylaminobenzyl)-6-(3-ethoxy-4-methoxyphenyl)-2,3,4,5-tetrahydropyridazin-3-one,
   and their physiologically acceptable salts and solvates;
   for preparing a medicament for treating myocardial diseases.

The preferred compounds show a selective inhibition of phosphodiesterase IV, which is associated with an intracellular increase in cAMP (N. Sommer et al., Nature Medicine, 1, 244-248 (1995)).

The inhibition of PDE IV can be demonstrated, for example, analogously to C. W. Davis in Biochim. Biophys. Acta 797, 354-362 (1984).

The affinity of the compounds of the invention for phosphodiesterase IV is measured by determining their IC₅₀ values (the concentration of inhibitor required to achieve 50% inhibition of the enzyme activity).

Preferably, the invention provides for the use of the compounds mentioned above for preparing a medicament for treating myocardial diseases, where said myocardial diseases show inflammatory and immunological characteristics.

Most preferably, the invention provides for the use of the compounds mentioned above for preparing a medicament for treating coronary artery disease, reversible or irreversible myocardial ischemia/reperfusion injury, acute or chronic heart failure and restenosis, including instent-restenosis and stent-in-stent-restenosis.

The preparations for the treatment of the mentioned diseases can be used as medicaments in human or veterinary medicine. Possible excipients are organic or inorganic substances which are suitable for enteral (e.g. oral) or parenteral administration or topical application and do not react with the novel compounds, for example water, vegetable oils, benzyl alcohols, alkylene glycols, polyethylene glycols, glycerol triacetate, gelatin, carbohydrates such as lactose or starch, magnesium stearate, talc and petroleum jelly. In particular, tablets, pills, coated tablets, capsules, powders, granules, syrups, juices or drops are used for oral administration, suppositories are used for rectal administration, solutions, preferably oily or aqueous solutions, and furthermore suspensions, emulsions or implants, are used for parenteral administration, and ointments, creams or powders are used for topical application. The novel compounds can also be lyophilized and the lyophilizates obtained used, for example, for the production of injection preparations. The preparations indicated can be sterilized and/or can contain auxiliaries such as lubricants, preservatives, stabilizers and/or wetting agents, emulsifiers, salts for affecting the osmotic pressure, buffer substances, colourants, flavourings and/or one or more further active compounds, e.g. one or more vitamins.

In these indications the substances are generally administered preferably in doses of between approximately 1 and 500 mg, in particular between 5 and 100 mg per dose unit. The daily dose is preferably between approximately 0.02 and 10 mg/kg of body weight. The specific dose for each patient depends, however, on a wide variety of factors, for example on the efficacy of the specific compound used, on age, body weight, general state of health, gender, on the diet, on the time and route of administration, on the excretion rate, medicament combination and severity of the particular disease to which the therapy is applied. Oral administration is preferred.

### EXAMPLE 1

### Effect of PDE4 Inhibitors on T-Cell Proliferation

Peripheral blood mononuclear cells (PBMC) were isolated from the blood of healthy donors by the Lymphoprep gradient method. 200000 PBMC/well were cultured in RPMI1640 culture medium containing 5% heat inactivated human serum (AB pool) for 5 days at 37° C. and 10% CO₂ in 96 well flat bottom microtiter plates. The T cells within the PBMC preparation were selectively stimulated with an monoclonal antibody to CD3. Cultures were set up as triplicates including a control group receiving no treatment. PDE4 inhibitors were dissolved in DMSO at 10⁻² M and diluted in culture medium. Control cultures were treated with DMSO equivalent to the inhibitor concentration. 18 hrs before the end of the assay, ³H-thymidine was added to the cultures. The incorporation of radioactivity into the cells was then measured in a beta-counter.

The data of at least three independent experiments were calculated as percent inhibition of the control (mean ±SEM) without inhibitor. From this data the IC-50 value was determined.

Results:

PDE4 inhibitors afforded a marked reduction of T-cell proliferation (see table 1).

TABLE 1
Effect of PDE4 inhibitors on T-cells and macrophages

| | IL2 | IFN-γ | TNF-α | IL10 | IL12 | |
|---|---|---|---|---|---|---|
| | T-cell | T-cell | macrophage | macrophage | macrophage | T-cell prol. |
| | IC₅₀ [μM] | IC₅₀ [μM] | IC₅₀ [μM] | EC₅₀ [μM] | IC₅₀ [μM] | IC₅₀ [μM] |
| Rolipram | 0.7 | 1 | 0.6 | 0.05 | >0.1 | 0.3 |
| EMD 94360 | 0.3 | 0.05 | 0.03 | 0.001 | 0.006 | 0.02 |
| EMD 95832 | 0.2 | 0.01 | 0.009 | 0.02 | 0.0005 | 0.05 |
| EMD 125025 | 0.002 | 0.007 | 0.003 | no effect | <1E−13M | 0.006 |
| EMD 125059 | 0.06 | 0.008 | 0.03 | 0.008 | <1E−13M | 0.04 |
| EMD 219901 | 0.003 | 0.005 | 0.002 | 0.001 | <1E−13M | 0.007 |

EMD 94360 (EP 0738715; Page 17, line 41):

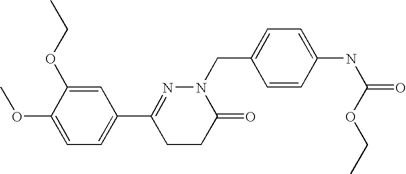

EMD 95832 (EP 0763534):

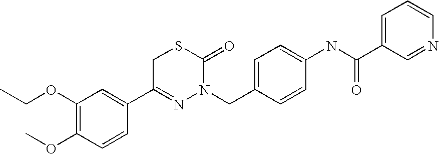

EMD 125025 (WO 98/06704; page 13, lines 16-17):

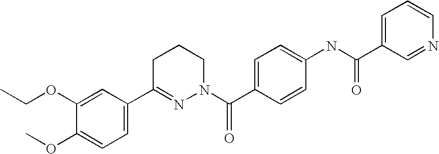

EMD 125059 (WO 98/06704; page 27, lines 2-3):

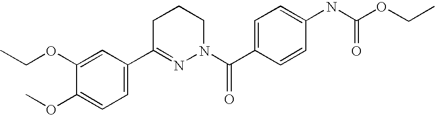

EMD 219901 (WO 99/65880; page 14, lines 20-21)

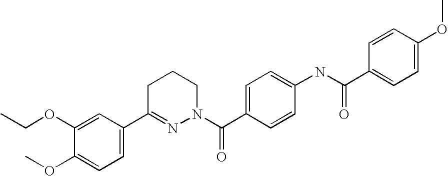

### EXAMPLE 2

### Effect of PDE4 Inhibitors on Cytokine Production in Human Peripheral Blood Monocytic Cells

Peripheral blood mononuclear cells (PBMC) were isolated from the blood of healthy donors by the Lymphoprep gradient method. 200000 PBMC/well were cultured in RPMI1640 culture medium containing 5% heat inactivated human serum (AS pool). at 37° C. and 10% CO₂ in 96 well flat bottom microtiter plates. Cultures were set up as triplicates including a control group. Solutions of PDE4 inhibitors were prepared in DMSO at 10⁻² M and diluted in culture medium. Control cultures were treated with concentrations of DMSO equivalent to the inhibitor concentrations. The cytokine of interest was stimulated as indicated in Table 2

The culture supernatants of three independent experiments were pooled and cytokine activity in the supernatant was measured with commercially available ELISA test kits.

TABLE 2
Activation of PBMC to form T-cell and
macrophage specific cytokines

| cytokine | activator | incubation in culture |
|---|---|---|
| IL-2 | mab anti-CD3 | 24 h |
| IL-10 | LPS | 48 h |
| TNF-α | LPS | 48 h |
| IFN-γ | mab anti-CD3 | 48 h |
| IL-12 | SAC + IFNγ | 48 h |

The data were calculated as percent inhibition/stimulation of the control without the compound and the IC₅₀ value or EC₅₀ value in case of stimulation was determined thereof.

Result

PDE4 inhibitors afforded a marked reduction in the release of IL-2, IFN-γ, TNF-α and IL-12. The immunosuppressant cytokine IL-10, however, was stimulated by most PDE4 inhibitors (see Table 1).

### EXAMPLE 3

### Effect of PDE4 Inhibitors on Experimental Myocardial Infarction in Rats

Compound 5, administered intraperitoneally with 1, 3, and 10 mg/kg, 1 hour before reversible occlusion of the left coronary artery in rats caused a significant dose dependent reduction of infarct size up to 38%. In correspondence with this protection, a reduction of plasma TNF-□ levels was observed, as measured by ELISA.

### EXAMPLE 4

### Effect of PDE4 Inhibitors on Experimental Myocardial Infarction in Rabbits

There was a cardioprotective effect by PDE4 inhibition in anaesthetised rabbits subjected to 30 minutes of coronary artery occlusion (side branch of the ramus circumflexus of the left coronary artery) followed by 120 minutes of reperfusion. PDE4 inhibitors applied prior to the coronary occlusion reduced infarct size as compared with placebo treatment. The areas at risk were comparable between verum and placebo groups. The cardioprotective effect cannot be attributed to favorable hemodynamic effects, since heart rate and mean aortic pressure remained constant throughout the experimental protocol.

1. Use of
a) compounds of formula I disclosed in EP 0763534 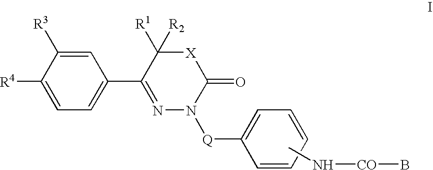
in which
B is an aromatic heterocycle having 1 to 4 N, O and/or S atoms, bonded via N or C, which can be unsubstituted or mono-, di- or trisubstituted by HaI, A and/or OA, and can also be fused to a benzene or pyridine ring,
Q is absent or is alkylene having 1-6 C atoms,
X is CH₂, S or O,
R¹ and R² in each case independently of one another are H or A,
R³ and R⁴ in each case independently of one another are —OH, OR⁵, —S—R⁵, —SO—R⁵, —SO₂—R⁵, HaI, methylenedioxy, —NO₂, —NH₂, —NHR⁵ or —NR⁵R⁶,
R⁵ and R⁶ in each case independently of one another are A, cycloalkyl having 3-7 C atoms, methylenecycloalkyl having 4-8 C atoms or alkenyl having 2-8 C atoms,
A is alkyl having 1 to 10 C atoms, which can be substituted by 1 to 5 F and/or Cl atoms and
HaI is F, Cl, Br or I
and their stereoisomers and physiologically acceptable, salts and solvates;
b) compounds of formula I disclosed in WO 99/65880 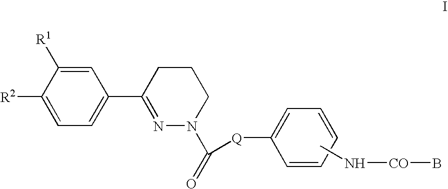
in which
B is a phenyl ring which is unsubstituted or mono- or polysubstituted by R³,
Q is absent or is alkylene having 1-4 C atoms,
R^{1,R}² each independently of one another are —OR⁴, —S—R⁴, —SO—R⁴, —SO₂—R⁴ or HaI,
R¹ and R² together are also —O—CH₂—O—,
R³ is R⁴, HaI, OH, OR⁴, OPh, NO₂, NHR⁴, N(R⁴)₂, NHCOR⁴, NHSO₂R⁴ or NHCOOR⁴,
R⁴ is A, cycloalkyl having 3-7 C atoms, alkylenecycloalkyl having 5-10 C atoms or alkenyl having 2-8 C atoms,
A is alkyl having 1 to 10 C atoms, which can be substituted by 1 to 5 F and/or Cl atoms and
HaI is F, Cl, Br or I,
and their physiologically acceptable salts and solvates;
c) compounds of formula I disclosed in WO 00/26201 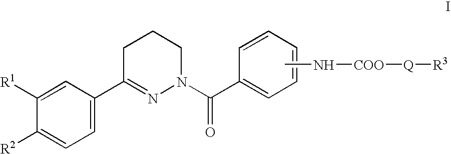
in which
R¹, R² in each case independently of one another are —OH, OR⁵, —S—R⁵, —SO—R⁵, —SO₂—R⁵ or HaI,
R¹ and R² together are also —O—CH₂—O—,
R³ is NH₂, NHA, NAA′ or a saturated heterocycle having 1 to 4 N, O and/or S atoms which can be unsubstituted or mono-, di- or tri-substituted by HaI, A and/or OA,
Q is absent or is branched or unbranched alkylene having 1-10 C atoms,
R⁵ is A, cycloalkyl having 3-7 C atoms, alkylenecycloalkyl having 4-8 C atoms or alkenyl having 2-8 C atoms,
A, A′ in each case independently of one another are alkyl which has 1 to 10 C atoms and which can be substituted by 1 to 5 F and/or Cl atoms and
HaI is F, Cl, Br or I,
and the physiologically acceptable salts and solvates thereof;
d) compounds of formula I disclosed in WO 98/06704 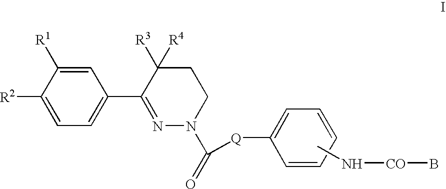
in which
B is A, OA, NH₂, NHA, NAA′ or an unsaturated heterocycle which has 1 to 4 N, O and/or S atoms and which can be unsubstituted or mono-, di- or trisubstituted by HaI, A and/or OA,
Q is absent or is alkylene having 1-6 C atoms,
R¹, R² in each case independently of one another are —OH, OR⁵, —S—R⁵, —SO—R⁵, —SO₂—R⁵, HaI, —NO₂, —NH₂, —NHR⁵ or —NRR₆,
R¹ and R² together are also —O—CH₂—O—,
R³, R⁴ in each case independently of one another are H or A,
R⁵, R⁶ in each case independently of one another are A, cycloalkyl having 3-7 C atoms, methylenecycloalkyl having 4-8 C atoms or alkenyl having 2-8 C atoms,
A, A′ in each case independently of one another are alkyl which has 1 to 10 C atoms and which can be substituted by 1 to 5 F and/or Cl atoms and
HaI is F, Cl, Br or I,
and the stereoisomers and physiologically acceptable salts and solvates thereof;
e) compounds disclosed in WO 00/59890
1-(4-ureidobenzoyl)-3-(3-ethoxy-4-methoxyphenyl)-1,4,5,6-tetrahydropyridazine,
1-(4-nicotinoylaminobenzoyl)-3-(3-propoxy-4-methoxyphenyl)-1,4,5,6-tetrahydropyridazine,
1-(4-trifluoroacetamidobenzoyl)-3-(3-ethoxy-4-methoxyphenyl)-1,4,5,6-tetrahydropyridazine,
1-(4-ethoxycarbonylaminobenzoyl)-3-(3-propoxy-4-methoxyphenyl)-1,4,5,6-tetrahydropyridazine,
1-(4-isopropoxycarbonylaminobenzoyl)-3-(3-ethoxy-4-methoxyphenyl)-1,4,5,6-tetrahydropyridazine,
1-(4-propoxycarbonylaminobenzoyl)-3-(3-ethoxy-4-methoxyphenyl)-1,4,5,6-tetrahydropyridazine,
1-(4-nicotinoylaminobenzoyl)-3-(3,4-dimethoxyphenyl)-4-ethyl-1,4,5,6-tetrahydropyridazine,
1-(4-ethoxycarbonylaminobenzoyl)-3-(3,4-dimethoxyphenyl)-4-ethyl-1,4,5,6-tetrahydropyridazine and
1-(4-acetamidobenzoyl)-3-(3,4-dimethoxyphenyl)-4-ethyl-1,4,5,6-tetrahydropyridazine,
and their physiologically acceptable salts and solvates;
f) compounds of formula I disclosed in DE 19604388 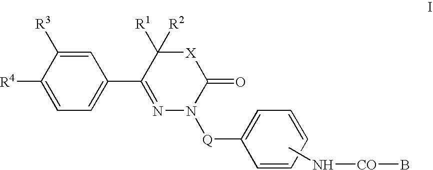
in which
R¹, R² in each case independently of one another are H or A,
R³, R⁴ in each case independently of one another are —OH, OA, —S-A, —SO-A, —SO₂-A, HaI, methylenedioxy, —NO₂, —NH₂, —NHA or —NAA′,
A, A′ in each case independently of one another are alkyl having 1 to 10 C-atoms, and which can be substituted by 1 to 5 F and/or Cl atoms, cycloalkyl having 3-7 C atoms or methylenecycloalkyl having 4-8 C atoms,
B is —Y—R⁵ oder —O—Y—R⁵,
Q is absent or is alkylene having 1-4 C atoms,
Y is absent or is alkylene having 1-10 C atoms,
X is CH₂or S,
R⁵ is NH₂, NHA, NAA′ or is a saturated 3-8 membered heterocycle having at least one N atom, and wherein other CH₂ groups optionally may be replaced by NH, NA, S or O, which can be unsubstituted or monosubstituted by A or OH,
HaI is F, Cl, Br oder I
and the stereoisomers and physiologically acceptable salts and solvates thereof;
g) compounds of formula I disclosed in DE 19932315 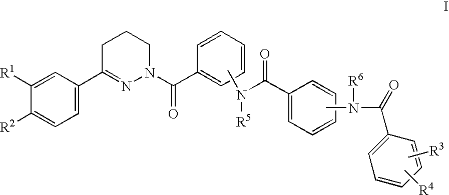
in which
R¹, R² in each case independently of one another are H, OH, OA, SA, SOA, SO₂A, F, Cl or A′₂N—(CH₂)ₙ—O—,
R¹ and R² together are also —O—CH₂—O—,
R³, R⁴ in each case independently of one another are H, A, HaI, OH, OA, NO₂, NHA, NA₂, CN, COOH, COOA, NHCOA, NHSO₂A or NHCOOA,
R⁵, R⁶ in each case independently of one another are H or alkyl having 1 to 6 C atoms,
A is alkyl having 1 to 10 C atoms, which can be substituted by 1 to 5 F and/or Cl atoms,
is cycloalkyl having 3-7 C atoms, alkylenecycloalkyl having 5-10 C atoms or alkenyl having 2-8 C atoms,
A′ is alkyl having 1, 2, 3, 4, 5 or 6 C atoms,
n is 1, 2, 3 or 4,
HaI is F, Cl, Br or I,
and their physiologically acceptable salts and solvates;
h) compounds of formula I disclosed in EP 0723962 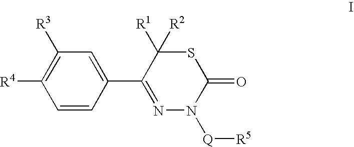
in which
R¹ and R² in each case independently of one another are H or A,
R³ and R⁴ in each case independently of one another are —OH, —OR¹⁰, —S—R¹⁰, —SO—R¹⁰, —SO₂R¹⁰, HaI, methylenedioxy, —NO₂, —NH₂, —NHR¹⁰ or —NR¹⁰R¹¹,
R⁵ is a phenyl radical which is unsubstituted or mono- or disubstituted by R⁶ and/or R⁷,
Q is absent or is alkylene having 1-6 C atoms,
R⁶ and R⁷ in each case independently of one another are —NH₂, —NR⁸R⁹, —NHR¹⁰, —NR¹⁰R¹¹, —NO₂, HaI, —CN, —OA, —COOH or —COOA,
R⁸ and R⁹ in each case independently of one another are H, acyl having 1-8 C atoms which can be substituted by 1-5 F and/or Cl atoms, —COOA, —S-A, —SO-A, —SO₂A, —CONH₂, —CONHA, —CONA₂, —CO—COOH, —CO—COOA, —CO—CONH₂, —CO—CONHA or —CO—CONA₂,
A is alkyl having 1 to 6 C atoms which can be substituted by 1-5 F and/or Cl atoms,
R¹⁰ and R¹¹ in each case independently of one another are A, cycloalkyl having 3-7 C atoms, methylenecycloalkyl having 4-8 C atoms or alkenyl having 2-8 C-atoms
and
HaI is F, Cl, Br or I,
and their physiologically acceptable salts and solvates;
i) compounds of formula I disclosed in EP 0738715 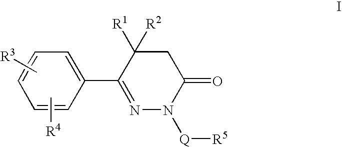
in which
R¹ and R² in each case independently of one another are H or A,
R³ and R⁴ in each case independently of one another are —OH, —OR¹⁰, —S—R¹⁰, —SO—R¹⁰, —SO₂R¹⁰, HaI, methylenedioxy, —NO₂, —NH₂, —NHR¹⁰ or —NR¹⁰R¹¹,
R⁵ is a phenyl radical which is unsubstituted or mono- or disubstituted by R⁶ and/or R⁷,
Q is absent or is alkylene having 1-6 C atoms,
R⁶ and R⁷ in each case independently of one another are —NH₂, —NR⁸R⁹, —NHR¹⁰, —NR¹⁰R¹¹, —NO₂, HaI, —CN, —OA, —COOH or —COOA,
R⁸ and R⁹ in each case independently of one another are H, acyl having 1-8 C atoms which can be substituted by 1-5 F and/or Cl atoms, —COOA, —SO-A, —SO₂A, —CONH₂, —CONHA, —CONA₂, —CO—COOH, —CO—COOA, —CO—CONH₂, —CO—CONHA or —CO—CONA₂,
A is alkyl having 1 to 6 C atoms which can be substituted by 1-5 F and/or Cl atoms,
R¹⁰ and R¹¹ in each case independently of one another are A, cycloalkyl having 3-7 C atoms, methylenecycloalkyl having 4-8 C atoms or alkenyl having 2-8 C-atoms
and
HaI is F, Cl, Br or I,
and their physiologically acceptable salts and solvates;
for preparing a medicament for treating myocardial diseases.

2. Use according to claim 1 of
a) compounds disclosed in EP 0763534:
2-(3-nicotinoylaminobenzyl)-6-(3,4-dimethoxyphenyl)-2,3,4,5-tetrahydropyridazin-3-one,
2-(2-nicotinoylaminobenzyl)-6-(3,4-dimethoxyphenyl)-2,3,4,5-tetrahydropyridazin-3-one,
2-(4-nicotinoylaminobenzyl)-6-(3,4-dimethoxyphenyl)-5-ethyl-2,3,4,5-tetrahydropyridazin-3-one,
2-(3-nicotinoylaminobenzyl)-6-(3,4-dimethoxyphenyl)-5-ethyl-2,3,4,5-tetrahydropyridazin-3-one,
2-(2-nicotinoylaminobenzyl)-6-(3,4-dimethoxyphenyl)-5-ethyl-2,3,4,5-tetrahydropyridazin-3-one,
2-(4-nicotinoylaminobenzyl)-6-(3-methoxy-4-trifluoromethoxyphenyl)-5-ethyl-2,3,4,5-tetrahydropyridazin-3-one,
2-(4-nicotinoylaminobenzyl)-6-(3-methoxy-4-difluoromethoxyphenyl)-5-ethyl-2,3,4,5-tetrahydropyridazin-3-one,
2-(4-nicotinoylaminobenzyl)-6-(3-methoxy-4-fluoromethoxyphenyl)-5-ethyl-2,3,4,5-tetrahydropyridazin-3-one,
2-(4-nicotinoylaminobenzyl)-6-(3-difluoromethoxy-4-methoxyphenyl)-5-ethyl-2,3,4,5-tetrahydropyridazin-3-one,
2-(4-nicotinoylaminobenzyl)-6-(3-trifluoromethoxy-4-methoxyphenyl)-5-ethyl-2,3,4,5-tetrahydropyridazin-3-one,
2-(4-nicotinoylaminobenzyl)-6-(3-fluoromethoxy-4-methoxyphenyl)-5-ethyl-2,3,4,5-tetrahydropyridazin-3-one,
2-(4-nicotinoylaminobenzyl)-6-(3-methoxy-4-ethoxyphenyl)-5-ethyl-2,3,4,5-tetrahydropyridazin-3-one,
2-(4-nicotinoylaminobenzyl)-6-(3-ethoxy-4-methoxyphenyl)-5-ethyl-2,3,4,5-tetrahydropyridazin-3-one,
2-(4-nicotinoylaminobenzyl)-6-(3-hydroxy-4-methoxyphenyl)-5-ethyl-2,3,4,5-tetrahydropyridazin-3-one,
2-(4-nicotinoylaminobenzyl)-6-(4-methylsulfonylphenyl)-5-ethyl-2,3,4,5-tetrahydropyridazin-3-one,
2-(4-nicotinoylaminobenzyl)-6-(4-methyleneoxyphenyl)-5-ethyl-2,3,4,5-tetrahydropyridazin-3-one,
2-(4-nicotinoylaminobenzyl)-6-(3-cyclopentyloxy-4-methoxyphenyl)-5-ethyl-2,3,4,5-tetrahydropyridazin-3-one,
2-(3-nicotinoylaminobenzyl)-6-(3-cyclopentyloxy-4-methoxyphenyl)-5-ethyl-2,3,4,5-tetrahydropyridazin-3-one,
2-(4-nicotinoylaminophenethyl)-6-(3,4-dimethoxyphenyl)-2,3,4,5-tetrahydropyridazin-3-one,
2-(4-nicotinoylaminophenethyl)-6-(3,4-dimethoxyphenyl)-5-ethyl-2,3,4,5-tetrahydropyridazin-3-one,
3-(4-nicotinoylaminobenzyl)-5-(3,4-dimethoxyphenyl)-3,6-dihydro-1,3,4-thiadiazin-2-one,
3-(3-nicotinoylaminobenzyl)-5-(3,4-dimethoxyphenyl)-3,6-dihydro-1,3,4-thiadiazin-2-one,
3-(2-nicotinoylaminobenzyl)-5-(3,4-dimethoxyphenyl)-3,6-dihydro-1,3,4-thiadiazin-2-one,
3-(4-nicotinoylaminobenzyl)-5-(3,4-dimethoxyphenyl)-6-ethyl-3,6-dihydro-1,3,4-thiadiazin-2-one,
3-(3-nicotinoylaminobenzyl)-5-(3,4-dimethoxyphenyl)-6-ethyl-3,6-dihydro-1,3,4-thiadiazin-2-one,
3-(2-nicotinoylaminobenzyl)-5-(3,4-dimethoxyphenyl)-6-ethyl-3,6-dihydro-1,3,4-thiadiazin-2-one,
3-(4-nicotinoylaminobenzyl)-5-(3-methoxy-4-trifluoromethoxyphenyl)-6-ethyl-3,6-dihydro-1,3,4-thiadiazin-2-one,
3-(4-nicotinoylaminobenzyl)-5-(3-methoxy-4-difluoromethoxyphenyl)-6-ethyl-3,6-dihydro-1,3,4-thiadiazin-2-one,
3-(4-nicotinoylaminobenzyl)-5-(3-methoxy-4-fluoromethoxyphenyl)-6-ethyl-3,6-dihydro-1,3,4-thiadiazin-2-one,
3-(4-nicotinoylaminobenzyl)-5-(3-difluoromethoxy-4-methoxyphenyl)-6-ethyl-3,6-dihydro-1,3,4-thiadiazin-2-one,
3-(4-nicotinoylaminobenzyl)-5-(3-trifluoromethoxy-4-methoxyphenyl)-6-ethyl-3,6-dihydro-1,3,4-thiadiazin-2-one,
3-(4-nicotinoylaminobenzyl)-5-(3-fluoromethoxy-4-methoxyphenyl)-6-ethyl-3,6-dihydro-1,3,4-thiadiazin-2-one,
3-(4-nicotinoylaminobenzyl)-5-(3-methoxy-4-ethoxyphenyl)-6-ethyl-3,6-dihydro-1,3,4-thiadiazin-2-one,
3-(4-nicotinoylaminobenzyl)-5-(3-ethoxy-4-methoxyphenyl)-6-ethyl-3,6-dihydro-1,3,4-thiadiazin-2-one,
3-(4-nicotinoylaminobenzyl)-5-(3-ethoxy-4-methoxyphenyl)-3,6-dihydro-1,3,4-thiadiazin-2-one,
3-(4-nicotinoylaminobenzyl)-5-(3-hydroxy-4-methoxyphenyl)-6-ethyl-3,6-dihydro-1,3,4-thiadiazin-2-one,
3-(4-nicotinoylaminobenzyl)-5-(4-methysulfonylphenyl)-6-ethyl-3,6-dihydro-1,3,4-thiadiazin-2-one,
3-(4-nicotinoylaminobenzyl)-5-(4-methyleneoxyphenyl)-6-ethyl-3,6-dihydro-1,3,4-thiadiazin-2-one,
3-(4-nicotinoylaminobenzyl)-5-(3-cyclopentyloxy-4-methoxyphenyl)-6-ethyl-3,6-dihydro-1,3,4-thiadiazin-2-one,
3-(3-nicotinoylaminobenzyl)-5-(3-cyclopentyloxy-4-methoxyphenyl)-6-ethyl-3,6-dihydro-1,3,4-thiadiazin-2-one,
3-(4-nicotinoylaminophenethyl)-5-(3,4-dimethoxyphenyl)-3,6-dihydro-1,3,4-thiadiazin-2-one,
3-(4-nicotinoylaminophenethyl)-5-(3,4-dimethoxyphenyl)-6-ethyl-3,6-dihydro-1,3,4-thiadiazin-2-one,
3-(4-nicotinoylaminobenzyl)-5-(3,4-dimethoxyphenyl)-3,6-dihydro-1,3,4-oxadiazin-2-one,
3-(3-nicotinoylaminobenzyl)-5-(3,4-dimethoxyphenyl)-3,6-dihydro-1,3,4-oxadiazin-2-one,
3-(2-nicotinoylaminobenzyl)-5-(3,4-dimethoxyphenyl)-3,6-dihydro-1,3,4-oxadiazin-2-one,
3-(4-nicotinoylaminobenzyl)-5-(3,4-dimethoxyphenyl)-6-ethyl-3,6-dihydro-1,3,4-oxadiazin-2-one,
3-(3-nicotinoylaminobenzyl)-5-(3,4-dimethoxyphenyl)-6-ethyl-3,6-dihydro-1,3,4-oxadiazin-2-one,
3-(2-nicotinoylaminobenzyl)-5-(3,4-dimethoxyphenyl)-6-ethyl-3,6-dihydro-1,3,4-oxadiazin-2-one,
3-(4-nicotinoylaminobenzyl)-5-(3-methoxy-4-trifluoromethoxyphenyl)-6-ethyl-3,6-dihydro-1,3,4-oxadiazin-2-one,
3-(4-nicotinoylaminobenzyl)-5-(3-methoxy-4-difluoromethoxyphenyl)-6-ethyl-3,6-dihydro-1,3,4-oxadiazin-2-one,
3-(4-nicotinoylaminobenzyl)-5-(3-methoxy-4-fluoromethoxyphenyl)-6-ethyl-3,6-dihydro-1,3,4-oxadiazin-2-one,
3-(4-nicotinoylaminobenzyl)-5-(3-difluoromethoxy-4-methoxyphenyl)-6-ethyl-3,6-dihydro-1,3,4-oxadiazin-2-one,
3-(4-nicotinoylaminobenzyl)-5-(3-trifluoromethoxy-4-methoxyphenyl)-6-ethyl-3,6-dihydro-1,3,4-oxadiazin-2-one,
3-(4-nicotinoylaminobenzyl)-5-(3-fluoromethoxy-4-methoxyphenyl)-6-ethyl-3,6-dihydro-1,3,4-oxadiazin-2-one,
3-(4-nicotinoylaminobenzyl)-5-(3-methoxy-4-ethoxyphenyl)-6-ethyl-3,6-dihydro-1,3,4-oxadiazin-2-one,
3-(4-nicotinoylaminobenzyl)-5-(3-ethoxy-4-methoxyphenyl)-6-ethyl-3,6-dihydro-1,3,4-oxadiazin-2-one,
3-(4-nicotinoylaminobenzyl)-5-(3-hydroxy-4-methoxyphenyl)-6-ethyl-3,6-dihydro-1,3,4-oxadiazin-2-one,
3-(4-nicotinoylaminobenzyl)-5-(4-methylsulfonylphenyl)-6-ethyl-3,6-dihydro-1,3,4-oxadiazin-2-one,
3-(4-nicotinoylaminobenzyl)-5-(4-methyleneoxyphenyl)-6-ethyl-3,6-dihydro-1,3,4-oxadiazin-2-one,
3-(4-nicotinoylaminobenzyl)-5-(3-cyclopentyloxy-4-methoxyphenyl)-6-ethyl-3,6-dihydro-1,3,4-oxadiazin-2-one,
3-(3-nicotinoylaminobenzyl)-5-(3-cyclopentyloxy-4-methoxyphenyl)-6-ethyl-3,6-dihydro-1,3,4-oxadiazin-2-one,
3-(4-nicotinoylaminophenethyl)-5-(3,4-dimethoxyphenyl)-3,6-dihydro-1,3,4-oxadiazin-2-one,
3-(4-nicotinoylaminophenethyl)-5-(3,4-dimethoxyphenyl)-6-ethyl-3,6-dihydro-1,3,4-oxadiazin-2-one,
2-(3-nicotinoylaminobenzyl)-6-(3-ethoxy-4-methoxyphenyl)-2,3,4,5-tetrahydropyridazin-3-one,
2-(4-isonicotinoylaminobenzyl)-6-(3-ethoxy-4-methoxyphenyl)-2,3,4,5-tetrahydropyridazin-3-one,
2-(4-pyrazinecarbonylaminobenzyl)-6-(3-ethoxy-4-methoxyphenyl)-2,3,4,5-tetrahydropyridazin-3-one,
2-(4-(isoxazole-5-carbonylamino)benzyl)-6-(3-ethoxy-4-methoxyphenyl)-2,3,4,5-tetrahydropyridazin-3-one,
2-(4-nicotinoylaminobenzyl)-6-(3-cyclopentyloxy-4-methoxyphenyl)-2,3,4,5-tetrahydropyridazin-3-one,
2-(4-nicotinoylaminobenzyl)-6-(3,4,-dimethoxyphenyl)-2,3,4,5-tetrahydropyridazin-3-one, hydrochloride,
and their stereoisomers and physiologically acceptable, salts and solvates;
b) compounds disclosed in WO 99/65880
N-(3-(3-ethoxy-4-methoxyphenyl)-1,4,5,6-tetrahydropyridazin-1-ylcarbonyl)phenyl)-4-methoxybenzoyl-3-carboxamide,
N-(3-(3-ethoxy-4-methoxyphenyl)-1,4,5,6-tetrahydropyridazin-1-ylcarbonyl)phenyl)-4-methylbenzoyl-3-carboxamide,
N-(3-(3-ethoxy-4-methoxyphenyl)-1,4,5,6-tetrahydropyridazin-1-ylcarbonyl)phenyl)benzoyl-3-carboxamide,
N-(3-(3-ethoxy-4-methoxyphenyl)-1,4,5,6-tetrahydropyridazin-1-ylcarbonyl)phenyl)-3,4-dichlorobenzoyl-3-carboxamide,
N-(3-(3-ethoxy-4-methoxyphenyl)-1,4,5,6-tetrahydropyridazin-1-ylcarbonyl)phenyl)-4-trifluoromethylbenzoyl-3-carboxamide,
N-(3-(3-ethoxy-4-methoxyphenyl)-1,4,5,6-tetrahydropyridazin-1-ylcarbonyl)phenyl)-3-chlorobenzoyl-3-carboxamide,
N-(3-(3-ethoxy-4-methoxyphenyl)-1,4,5,6-tetrahydropyridazin-1-ylcarbonyl)phenyl)-4-fluorobenzoyl-3-carboxamide,
N-(3-(3-ethoxy-4-methoxyphenyl)-1,4,5,6-tetrahydropyridazin-1-ylcarbonyl)phenyl)-4-butoxybenzoyl-3-carboxamide,
N-(3-(3-ethoxy-4-methoxyphenyl)-1,4,5,6-tetrahydropyridazin-1-ylcarbonyl)phenyl)-4-pentoxybenzoyl-3-carboxamide,
N-(3-(3-ethoxy-4-methoxyphenyl)-1,4,5,6-tetrahydropyridazin-1-ylcarbonyl)phenyl)-4-ethoxybenzoyl-3-carboxamide,
N-(3-(3-ethoxy-4-methoxyphenyl)-1,4,5,6-tetrahydropyridazin-1-ylcarbonyl)phenyl)-3,4-dimethoxybenzoyl-3-carboxamide,
N-(3-(3-ethoxy-4-methoxyphenyl)-1,4,5,6-tetrahydropyridazin-1-ylcarbonyl)phenyl)-3-methylbenzoyl-3-carboxamide,
N-(3-(3-ethoxy-4-methoxyphenyl)-1,4,5,6-tetrahydropyridazin-1-ylcarbonyl)phenyl)-3-methoxybenzoyl-3-carboxamide,
and their physiologically acceptable salts and solvates;
c) compounds disclosed in WO 00/26201
3-dimethylaminopropyl{4-[3-(3-ethoxy-4-methoxyphenyl)-1,2,3,4-tetrahydropyridazin-1-ylcarbonyl]phenyl}carbamate,
N-methylpiperidin-4-yl-{4-[3-(3-ethoxy-4-methoxyphenyl)-1,2,3,4-tetrahydropyridazin-1-ylcarbonyl]phenyl}carbamate,
3-dimethylaminopropyl{4-[3-(3-isopropoxy-4-methoxyphenyl)-1,2,3,4-tetrahydropyridazin-1-ylcarbonyl]phenyl}carbamate,
3-dimethylaminopropyl{3-[3-(3-ethoxy-4-methoxyphenyl)-1,2,3,4-tetrahydropyridazin-1-ylcarbonyl]phenyl}carbamate,
3-dimethylaminopropyl{3-[3-(3-cyclopentyloxy-4-methoxyphenyl)-1,2,3,4-tetrahydropyridazin-1-ylcarbonyl]phenyl}carbamate,
N-methylpiperidin-4-yl-{3[3-(3-cyclopentyloxy-4-methoxyphenyl)-1,2,3,4-tetrahydropyridazin-1-ylcarbonyl]phenyl}carbamate,
3-dimethylaminopropyl{3-[3-(3-propyloxy-4-methoxyphenyl)-1,2,3,4-tetrahydropyridazin-1-ylcarbonyl]phenyl}carbamate,
3-dimethylaminopropyl{4-[3-(3,4-diethoxyphenyl)-1,2,3,4-tetrahydropyridazin-1-ylcarbonyl]phenyl}carbamate,
N-methylpiperidin-4-yl-{4-[3-(3,4-diethoxyphenyl)-1,2,3,4-tetrahydropyridazin-1-ylcarbonyl]phenyl}carbamate,
3-dimethylaminopropyl{3-[3-(3,4-dimethoxyphenyl)-1,2,3,4-tetrahydropyridazin-1-ylcarbonyl]phenyl}carbamate
3-dimethylaminopropyl{4-[3-(3,4-dimethoxyphenyl)-1,2,3,4-tetrahydropyridazin-1-ylcarbonyl]phenyl}carbamate,
and the physiologically acceptable salts and solvates thereof;
d) compounds disclosed in WO 98/06704
1-(4-nicotinoylaminobenzoyl)-3-(3,4-dimethoxyphenyl)-1,4,5,6-tetrahydropyridazine,
1-(3-nicotinoylaminobenzoyl)-3-(3,4-dimethoxy-phenyl)-1,4,5,6-tetrahydropyridazine hydrochloride,
1-(2-nicotinoylaminobenzoyl)-3-(3,4-dimethoxy-phenyl)-1,4,5,6-tetrahydropyridazine,
1-(4-nicotinoylaminobenzoyl)-3-(3-ethoxy-4-methoxyphenyl)-1,4,5,6-tetrahydropyridazine,
1-(3-nicotinoylaminobenzoyl)-3-(3-ethoxy-4-methoxyphenyl)-1,4,5,6-tetrahydropyridazine,
1-(4-nicotinoylaminobenzoyl)-3-(3-cyclopentyloxy-4-methoxyphenyl)-1,4,5,6-tetrahydropyridazine,
1-(3-nicotinoylaminobenzoyl)-3-(3-cyclopentyloxy-4-methoxyphenyl)-1,4,5,6-tetrahydropyridazine,
1-(4-nicotinoylaminobenzoyl)-3-(3,4-methylene-dioxyphenyl)-1,4,5,6-tetrahydropyridazine,
1-(4-nicotinoylaminobenzoyl)-3-(3-methoxy-4-methylsulfonylphenyl)-1,4,5,6-tetrahydro-pyridazine,
1-(4-nicotinoylaminobenzoyl)-3-(3-trifluoro-methoxy-4-methoxyphenyl)-1,4,5,6-tetrahydro-pyridazine,
1-(4-ethoxy-carbonylaminobenzoyl)-3-(3,4-dimethoxyphenyl)-1,4,5,6-tetrahydropyridazine,
1-(3-ethoxycarbonylaminobenzoyl)-3-(3,4-dimethoxy-phenyl)-1,4,5,6-tetrahydropyridazine,
1-(2-ethoxycarbonylaminobenzoyl)-3-(3,4-dimethoxy-phenyl)-1,4,5,6-tetrahydropyridazine,
1-(4-ethoxycarbonylaminobenzoyl)-3-(3-ethoxy-4-methoxyphenyl)-1,4,5,6-tetrahydropyridazine,
1-(3-ethoxycarbonylaminobenzoyl)-3-(3-ethoxy-4-methoxyphenyl)-1,4,5,6-tetrahydropyridazine,
1-(4-ethoxycarbonylaminobenzoyl)-3-(3-cyclo-pentyloxy-4-methoxyphenyl)-1,4,5,6-tetrahydro-pyridazine,
1-(3-ethoxycarbonylaminobenzoyl)-3-(3-cyclo-pentyloxy-4-methoxyphenyl)-1,4,5,6-tetrahydro-pyridazine,
1-(4-ethoxycarbonylaminobenzoyl)-3-(3,4-methylene-dioxyphenyl)-1,4,5,6-tetrahydropyridazine,
1-(4-ethoxycarbonylaminobenzoyl)-3-(3-methoxy-4-methylsulfonylphenyl)-1,4,5,6-tetrahydro-pyridazine,
1-(4-ethoxycarbonylaminobenzoyl)-3-(3-trifluoro-methoxy-4-methoxyphenyl)-1,4,5,6-tetrahydro-pyridazine,
and the stereoisomers and physiologically acceptable salts and solvates thereof;
e) compounds disclosed in EP 0723962
3-(4-ethoxycarbonylaminobenzyl)-5-(3-ethoxy-4-methoxyphenyl)-3,6-dihydro-1,3,4-thiadiazin-2-one,
3-(4-ethoxycarbonylaminobenzyl)-5-(3-cyclopentyloxy-4-methoxyphenyl)-3,6-dihydro-1,3,4-thiadiazin-2-one,
and their physiologically acceptable salts and solvates;
f) compounds disclosed in EP 0738715
2-(4-butyrylaminobenzyl)-6-(3,4-dimethoxyphenyl)-2,3,4,5-tetrahydropyridazin-3-one,
2-(4-acetamidobenzyl)-6-(3,4-dimethoxyphenyl)-2,3,4,5-tetrahydropyridazin-3-one,
2-(4-trifluoroacetamidobenzyl)-6-(3,4-dimethoxyphenyl)-2,3,4,5-tetrahydropyridazin-3-one,
2-(4-methylsulfonamidobenzyl)-6-(3,4-dimethoxyphenyl)-2,3,4,5-tetrahydropyridazin-3-one,
2-(4-propionylaminobenzyl)-6-(3,4-dimethoxyphenyl)-2,3,4,5-tetrahydropyridazin-3-one,
2-(4-tert-butylcarbonylaminobenzyl)-6-(3,4-dimethoxyphenyl)-2,3,4,5-tetrahydropyridazin-3-one,
2-(4-isobutyrylaminobenzyl)-6-(3,4-dimethoxyphenyl)-2,3,4,5-tetrahydropyridazin-3-one,
2-(4-methoxycarbonylaminobenzyl)-6-(3,4-dimethoxyphenyl)-2,3,4,5-tetrahydropyridazin-3-one,
2-(4-pivalylaminobenzyl)-6-(3,4-dimethoxyphenyl)-2,3,4,5-tetrahydropyridazin-3-one,
2-(4-cyclopentylcarbamoylbenzyl)-6-(3,4-dimethoxyphenyl)-2,3,4,5-tetrahydropyridazin-3-one,
2-(4-ethoxycarbonylaminobenzyl)-6-(3,4-dimethoxyphenyl)-2,3,4,5-tetrahydropyridazin-3-one,
2-(4-methoxalylaminobenzyl)-6-(3,4-dimethoxyphenyl)-2,3,4,5-tetrahydropyridazin-3-one,
2-(4-ureidobenzyl)-6-(3,4-dimethoxyphenyl)-2,3,4,5-tetrahydropyridazin-3-one,
2-(4-pentanoylaminobenzyl)-6-(3,4-dimethoxyphenyl)-2,3,4,5-tetrahydropyridazin-3-one,
2-(4-hexanoylaminobenzyl)-6-(3,4-dimethoxyphenyl)-2,3,4,5-tetrahydropiridazin-3-one,
2-(4-pentafluoropropionylaminobenzyl)-6-(3,4-dimethoxyphenyl)-2,3,4,5-tetrahydropyridazin-3-one,
2-(4-acetamidobenzyl)-6-(3,4-dimethoxyphenyl)-5-ethyl-2,3,4,5-tetrahydropyridazin-3-one,
2-(4-trifluoroacetamidobenzyl)-6-(3,4-dimethoxyphenyl)-5-ethyl-2,3,4,5-tetrahydropyridazin-3-one,
2-(4-methylsulfonamidobenzyl)-6-(3,4-dimethoxyphenyl)-5-ethyl-2,3,4,5-tetrahydropyridazin-3-one,
2-(4-propionylaminobenzyl)-6-(3,4-dimethoxyphenyl)-5-ethyl-2,3,4,5-tetrahydropyridazin-3-one,
2-(4-tert-butylcarbonylaminobenzyl)-6-(3,4-dimethoxyphenyl)-5-ethyl-2,3,4,5-tetrahydropyridazin-3-one,
2-(4-butyrylaminobenzyl)-6-(3,4-dimethoxyphenyl)-5-ethyl-2,3,4,5-tetrahydropyridazin-3-one,
2-(4-isobutyrylaminobenzyl)-6-(3,4-dimethoxyphenyl)-5-ethyl-2,3,4,5-tetrahydropyridazin-3-one,
2-(4-methoxycarbonylaminobenzyl)-6-(3,4-dimethoxyphenyl)-5-ethyl-2,3,4,5-tetrahydropyridazin-3-one,
2-(4-pivalylaminobenzyl)-6-(3,4-dimethoxyphenyl)-5-ethyl-2,3,4,5-tetrahydropyridazin-3-one,
2-(4-cyclopentylcarbamoylbenzyl)-6-(3,4-dimethoxyphenyl)-5-ethyl-2,3,4,5-tetrahydropyridazin-3-one,
2-(4-ethoxycarbonylaminobenzyl)-6-(3,4-dimethoxyphenyl)-5-ethyl-2,3,4,5-tetrahydropyridazin-3-one,
2-(4-methoxalylaminobenzyl)-6-(3,4-dimethoxyphenyl)-5-ethyl-2,3,4,5-tetrahydropyridazin-3-one,
2-(4-ureidobenzyl)-6-(3,4-dimethoxyphenyl)-5-ethyl-2,3,4,5-tetrahydropyridazin-3-one,
2-(4-pentanoylaminobenzyl)-6-(3,4-dimethoxyphenyl)-5-ethyl-2,3,4,5-tetrahydropyridazin-3-one,
2-(4-hexanoylaminobenzyl)-6-(3,4-dimethoxyphenyl)-5-ethyl-2,3,4,5-tetrahydropyridazin-3-one,
2-(4-pentafluoropropionylaminobenzyl)-6-(3,4-dimethoxyphenyl)-5-ethyl-2,3,4,5-tetrahydropyridazin-3-one,
2-(4-acetamidobenzyl)-6-(3-ethoxy-4-methoxyphenyl)-5-ethyl-2,3,4,5-tetrahydropyridazin-3-one,
2-(4-trifluoroacetamidobenzyl)-6-(3-ethoxy-4-methoxyphenyl)-5-ethyl-2,3,4,5-tetrahydropyridazin-3-one,
2-(4-methylsulfonamidobenzyl)-6-(3-ethoxy-4-methoxyphenyl)-5-ethyl-2,3,4,5-tetrahydropyridazin-3-one,
2-(4-propionylaminobenzyl)-6-(3-ethoxy-4-methoxyphenyl)-5-ethyl-2,3,4,5-tetrahydropyridazin-3-one,
2-(4-butyrylaminobenzyl)-6-(3-ethoxy-4-methoxyphenyl)-5-ethyl-2,3,4,5-tetrahydropyridazin-3-one,
2-(4-isobutyrylaminobenzyl)-6-(3-ethoxy-4-methoxyphenyl)-5-ethyl-2,3,4,5-tetrahydropyridazin-3-one,
2-(4-methoxycarbonylaminobenzyl)-6-(3-ethoxy-4-methoxyphenyl)-5-ethyl-2,3,4,5-tetrahydropyridazin-3-one,
2-(4-pivalylaminobenzyl)-6-(3-ethoxy-4-methoxyphenyl)-5-ethyl-2,3,4,5-tetrahydropyridazin-3-one,
2-(4-cyclopentylcarbamoylbenzyl)-6-(3-ethoxy-4-methoxyphenyl)-5-ethyl-2,3,4,5-tetrahydropyridazin-3-one,
2-(4-ethoxycarbonylaminobenzyl)-6-(3-ethoxy-4-methoxyphenyl)-5-ethyl-2,3,4, 5-tetrahydropyridazin-3-one;
2-(4-methoxalylaminobenzyl)-6-(3-ethoxy-4-methoxyphenyl)-5-ethyl-2,3,4,5-tetrahydropyridazin-3-one,
2-(4-ureidobenzyl)-6-(3-ethoxy-4-methoxyphenyl)-5-ethyl-2,3,4,5-tetrahydropyridazin-3-one,
2-(4-pentanoylaminobenzyl)-6-(3-ethoxy-4-methoxyphenyl)-5-ethyl-2,3,4,5-tetrahydropyridazin-3-one,
2-(4-hexanoylaminobenzyl)-6-(3-ethoxy-4-methoxyphenyl)-5-ethyl-2,3,4,5-tetrahydropyridazin-3-one,
2-(4-pentafluoropropionylaminobenzyl)-6-(3-ethoxy-4-methoxyphenyl)-5-ethyl-2,3,4,5-tetrahydropyridazin-3-one,
2-(4-acetamidobenzyl)-6-(3-cyclopentyloxy-4-methoxyphenyl)-5-ethyl-2,3,4,5-tetrahydropyridazin-3-one,
2-(4-trifluoroacetamidobenzyl)-6-(3-cyclopentyloxy-4-methoxyphenyl)-5-ethyl-2,3,4,5-tetrahydropyridazin-3-one,
2-(4-methylsulfonamidobenzyl)-6-(3-cyclopentyloxy-4-methoxyphenyl)-5-ethyl-2,3,4,5-tetrahydropyridazin-3-one,
2-(4-propionylaminobenzyl)-6-(3-cyclopentyloxy-4-methoxyphenyl)-5-ethyl-2,3,4,5-tetrahydropyridazin-3-one,
2-(4-tert-butylcarbonylaminobenzyl)-6-(3-cyclopentyloxy-4-methoxyphenyl)-5-ethyl-2,3,4,5-tetrahydropyridazin-3-one,
2-(4-butyrylaminobenzyl)-6-(3-cyclopentyloxy-4-methoxyphenyl)-5-ethyl-2,3,4,5-tetrahydropyridazin-3-one,
2-(4-isobutyrylaminobenzyl)-6(3-cyclopentyloxy-4-methoxyphenyl)-5-ethyl-2,3,4,5-tetrahydropyridazin-3-one,
2-(4-methoxycarbonylaminobenzyl)-6-(3-cyclopentyloxy-4-methoxyphenyl)-5-ethyl-2,3,4,5-tetrahydropyridazin-3-one,
2-(4-pivalylaminobenzyl)-6-(3-cyclopentyloxy-4-methoxyphenyl)-5-ethyl-2,3,4,5-tetrahydropyridazin-3-one,
2-(4-cyclopentylcarbamoylbenzyl)-6-(3-cyclopentyloxy-4-methoxyphenyl)-5-ethyl-2,3,4,5-tetrahydropyridazin-3-one,
2-(4-ethoxycarbonylaminobenzyl)-6-(3-cyclopentyloxy-4-methoxyphenyl)-5-ethyl-2,3,4,5-tetrahydropyridazin-3-one,
2-(4-methoxalylaminobenzyl)-6-(3-cyclopentyloxy-4-methoxyphenyl)-5-ethyl-2,3,4,5-tetrahydropyridazin-3-one,
2-(4-ureidobenzyl)-6-(3-cyclopentyloxy-4-methoxyphenyl)-5-ethyl-2,3,4,5-tetrahydropyridazin-3-one,
2-(4-pentanoylaminobenzyl)-6-(3-cyclopentyloxy-4-methoxyphenyl)-5-ethyl-2,3,4,5-tetrahydropyridazin-3-one,
2-(4-hexanoylaminobenzyl)-6-(3-cyclopentyloxy-4-methoxyphenyl)-5-ethyl-2,3,4,5-tetrahydropyridazin-3-one,
2-(4-pentafluoropropionylaminobenzyl)-6-(3-cyclopentyloxy-4-methoxyphenyl)-5-ethyl-2,3,4,5-tetrahydropyridazin-3-one,
2-(4-acetamidobenzyl)-6-(3-ethoxy-4-methoxyphenyl)-2,3,4,5-tetrahydropyridazin-3-one,
2-(4-trifluoroacetamidobenzyl)-6-(3-ethoxy-4-methoxyphenyl)-2,3,4,5-tetrahydropyridazin-3-one,
2-(4-methylsulfonamidobenzyl)-6-(3-ethoxy-4-methoxyphenyl)-2,3,4,5-tetrahydropyridazin-3-one,
2-(4-propionylaminobenzyl)-6-(3-ethoxy-4-methoxyphenyl)-2,3,4,5-tetrahydropyridazin-3-one,
2-(4-butyrylaminobenzyl)-6-(3-ethoxy-4-methoxyphenyl)-2,3,4,5-tetrahydropyridazin-3-one,
2-(4-isobutyrylaminobenzyl)-6-(3-ethoxy-4-methoxyphenyl)-2,3,4,5-tetrahydropyridazin-3-one,
2-(4-methoxycarbonylaminobenzyl)-6-(3-ethoxy-4-methoxyphenyl)-2,3,4,5-tetrahydropyridazin-3-one,
2-(4-pivalylaminobenzyl)-6-(3-ethoxy-4-methoxyphenyl)-2,3,4,5-tetrahydropyridazin-3-one,
2-(4-cyclopentylcarbamoylbenzyl)-6-(3-ethoxy-4-methoxyphenyl)-2,3,4,5-tetrahydropyridazin-3-one,
2-(4-ethoxycarbonylaminobenzyl)-6-(3-ethoxy-4-methoxyphenyl)-2,3,4,5-tetrahydropyridazin-3-one,
2-(4-methoxalylaminobenzyl)-6-(3-ethoxy-4-methoxyphenyl)-2,3,4,5-tetrahydropyridazin-3-one,
2-(4-ureidobenzyl)-6-(3-ethoxy-4-methoxyphenyl)-2,3,4,5-tetrahydropyridazin-3-one,
2-(4-pentanoylaminobenzyl)-6-(3-ethoxy-4-methoxyphenyl)-2,3,4,5-tetrahydropyridazin-3-one,
2-(4-hexanoylaminobenzyl)-6-(3-ethoxy-4-methoxyphenyl)-2,3,4,5-tetrahydropyridazin-3-one,
2-(4-pentafluoropropionylaminobenzyl)-6-(3-ethoxy-4-methoxyphenyl)-2,3,4,5-tetrahydropyridazin-3-one,
and their physiologically acceptable salts and solvates;
for preparing a medicament for treating myocardial diseases.

3. Use according to claim 1 of compounds selected from
3-(4-nicotinoylaminobenzyl)-5-(3-ethoxy-4-methoxyphenyl)-3,6-dihydro-1,3,4-thiadiazin-2-one,
N-(3-(3-ethoxy-4-methoxyphenyl)-1,4,5,6-tetrahydropyridazin-1-ylcarbonyl)phenyl)-4-methoxybenzoyl-3-carboxamide,
1-(4-nicotinoylaminobenzoyl)-3-(3-ethoxy-4-methoxyphenyl)-1,4,5,6-tetrahydropyridazine,
1-(4-ethoxycarbonylaminobenzoyl)-3-(3-ethoxy-4-methoxyphenyl)-1,4,5,6-tetrahydropyridazine,
2-(4-ethoxycarbonylaminobenzyl)-6-(3-ethoxy-4-methoxyphenyl)-2,3,4,5-tetrahydropyridazin-3-one,
and their physiologically acceptable salts and solvates;
for preparing a medicament for treating myocardial diseases.

4. Use according to claim 1 for preparing a medicament for treating myocardial diseases, where said myocardial diseases show inflammatory and immunological characteristics.

5. Use according to claim 1 for preparing a medicament for treating coronary artery disease, reversible or irreversible myocardial ischemia/reperfusion injury, acute or chronic heart failure and restenosis, including instent-restenosis and stent-in-stent-restenosis.
